(19) United States
(12) Patent Application Publication
(10) Pub. No.: US 2026/0115032 A1
(43) Pub. Date: Apr. 30, 2026

(54) GAIT MODIFICATION SYSTEMS AND METHODS

(71) Applicant: Scientific Motion Technologies, Inc., Orlando, FL (US)

(72) Inventors: Peter RUNNING, Missoula, MT (US); Donald RUNNING, Missoula, MT (US); Daniel F. JUSTIN, Orlando, FL (US); Tom ASTLEY, Buckinghamshire (GB); R. John DOUGALL, York (GB)

(21) Appl. No.: 19/370,658

(22) Filed: Oct. 27, 2025

Related U.S. Application Data
(60) Provisional application No. 63/712,422, filed on Oct. 26, 2024.

(51) Int. Cl. A61F 5/14; A43B 7/22

(52) U.S. Cl. CPC A61F 5/14; A43B 7/22

(57) ABSTRACT

A system may be provided for modifying gait mechanics of a patient. The system may include a shoe with a first outsole, and a second outsole configured to attach to the first outsole. The second outsole may have a hindfoot portion, a forefoot portion, and a central portion connecting the hindfoot portion and the forefoot portion. The hindfoot portion may be adjustably positionable relative to the first outsole independently of the forefoot portion. The forefoot portion may be adjustably positionable relative to the first outsole independently of the hindfoot portion.

### CROSS REFERENCE TO RELATED APPLICATIONS

This application claims the benefit of U.S. Provisional Application Ser. No. 63/712,422, filed on Oct. 26, 2024 and entitled GAIT MODIFICATION SYSTEMS AND METHODS, the disclosure of which is incorporated as though set forth herein in its entirety.

### TECHNICAL FIELD

The present disclosure relates to systems and methods for use in gait optimization therapy. More specifically, the present disclosure relates to systems and methods for modifying gait mechanics using footwear.

### BACKGROUND

Gait optimization therapy plays a vital role in addressing mobility issues caused by various orthopedic conditions, many of which result in abnormal walking patterns that can worsen over time without intervention. Gait refers to the coordinated movement of the body during walking, and disruptions in this process can lead to inefficiency, discomfort, and long-term joint deterioration. For individuals suffering from orthopedic conditions, gait abnormalities often stem from issues with joint alignment, muscular imbalances, or compensatory movements designed to reduce pain. Without proper therapeutic intervention, these abnormalities can further aggravate joint pain and contribute to chronic mobility problems.

A variety of orthopedic conditions necessitate gait optimization therapy, particularly those that affect joint function and alignment. Osteoarthritis, a common degenerative joint disease, often leads to joint stiffness, swelling, and pain, which cause individuals to modify their gait in an attempt to reduce discomfort. This maladaptive walking pattern can result in uneven weight distribution, improper joint alignment, and increased stress on unaffected joints, accelerating wear and tear. Rheumatoid arthritis, which inflames joints, has a similar impact on gait mechanics, with patients adopting compensatory walking strategies that exacerbate joint misalignment and pain over time.

Traditional therapeutic interventions for correcting gait abnormalities in orthopedic patients include the use of static orthotics, physical therapy, and in some cases, surgery. However, conventional orthotic devices are often limited by their inability to adjust to the dynamic changes in a patient's gait during rehabilitation. Additionally, many of these solutions provide fixed support, which may not be optimal as the patient's condition evolves.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings.

FIG. 1 is a bottom perspective view of a gait modifier system according to an embodiment of the present disclosure.

FIG. 2A is a bottom view of a first gait modifier system and a second gait modifier system according to an embodiment of the present disclosure.

FIG. 2B is a perspective view of a gait modifier shoe according to an embodiment of the present disclosure.

FIG. 3A is a side view of a gait modifier outsole according to an embodiment of the present disclosure.

FIG. 3B. is a perspective view of a gait modifier outsole according to an embodiment of the present disclosure.

FIG. 3C is a sketch of alternate slot patterns of a gait modifier outsole according to an embodiment of the present disclosure.

FIG. 3D is an exploded perspective view of a gait modifier according to an embodiment of the present disclosure.

FIG. 3E is a perspective view of the gait modifier of FIG. 3D.

FIG. 3F is a perspective view of the gait modifier of FIG. 3D.

FIG. 3G is a bottom view of the gait modifier of FIG. 3D.

FIG. 3H is a side view of the gait modifier of FIG. 3D.

FIG. 4A is a perspective view of a small transition directional gait modifier outsole according to an embodiment of the present disclosure.

FIG. 4B is a bottom view of the small transition directional gait modifier outsole of FIG. 4A.

FIG. 4C is a side view of the small transition directional gait modifier outsole of FIG. 4A.

FIG. 5A is a perspective view of a small transition omni gait modifier outsole according to an embodiment of the present disclosure.

FIG. 5B is a bottom view of the small transition omni gait modifier outsole of FIG. 5A.

FIG. 5C is a side view of the small transition omni gait modifier outsole of FIG. 5A.

FIG. 6A is a perspective view of a small transition hybrid gait modifier outsole according to an embodiment of the present disclosure.

FIG. 6B is a bottom view of the small transition hybrid gait modifier outsole of FIG. 6A.

FIG. 6C is a side view of the small transition hybrid gait modifier outsole of FIG. 6A.

FIG. 7A is a perspective view of a small flat directional gait modifier outsole according to an embodiment of the present disclosure.

FIG. 7B is a bottom view of the small flat directional gait modifier outsole of FIG. 7A.

FIG. 7C is a side view of the small flat directional gait modifier outsole of FIG. 7A.

FIG. 8A is a perspective view of a small flat omni gait modifier outsole according to an embodiment of the present disclosure.

FIG. 8B is a bottom view of the small flat omni gait modifier outsole of FIG. 8A.

FIG. 8C is a side view of the small flat omni gait modifier outsole of FIG. 8A.

FIG. 9A is a perspective view of a small flat hybrid gait modifier outsole according to an embodiment of the present disclosure.

FIG. 9B is a bottom view of the small flat hybrid gait modifier outsole of FIG. 9A.

FIG. 9C is a side view of the small flat hybrid gait modifier outsole of FIG. 9A.

FIG. 10A is a perspective view of a large transition directional gait modifier outsole according to an embodiment of the present disclosure.

FIG. 10B is a bottom view of the large transition directional gait modifier outsole of FIG. 10A.

FIG. 10C is a side view of the large transition directional gait modifier outsole of FIG. 10A.

FIG. 11A is a perspective view of a large transition omni gait modifier outsole according to an embodiment of the present disclosure.

FIG. 11B is a bottom view of the large transition omni gait modifier outsole of FIG. 11A.

FIG. 11C is a side view of the large transition omni gait modifier outsole of FIG. 11A.

FIG. 12A is a perspective view of a large transition hybrid gait modifier outsole according to an embodiment of the present disclosure.

FIG. 12B is a bottom view of the large transition hybrid gait modifier outsole of FIG. 12A.

FIG. 12C is a side view of the large transition hybrid gait modifier outsole of FIG. 12A.

FIG. 13A is a perspective view of a large flat directional gait modifier outsole according to an embodiment of the present disclosure.

FIG. 13B is a bottom view of the large flat directional gait modifier outsole of FIG. 13A.

FIG. 13C is a side view of the large flat directional gait modifier outsole of FIG. 13A.

FIG. 14A is a perspective view of a large flat omni gait modifier outsole according to an embodiment of the present disclosure.

FIG. 14B is a bottom view of the large flat omni gait modifier outsole of FIG. 14A.

FIG. 14C is a side view of the large flat omni gait modifier outsole of FIG. 14A.

FIG. 15A is a perspective view of a large flat hybrid gait modifier outsole according to an embodiment of the present disclosure.

FIG. 15B is a bottom view of the large flat hybrid gait modifier outsole of FIG. 15A.

FIG. 15C is a side view of the large flat hybrid gait modifier outsole of FIG. 15A.

FIG. 16A is a perspective view of a gait modifier shoe according to an embodiment of the present disclosure.

FIG. 16B includes top and bottom views of a plate and a bottom view of a gait modifier outsole according to an embodiment of the present disclosure.

FIG. 17A is a perspective view of a gait modifier outsole according to an embodiment of the present disclosure.

FIG. 17B is a perspective view of a plate according to an embodiment of the present disclosure.

FIG. 17C is a perspective view of a gait modifier according to an embodiment of the present disclosure.

FIG. 17D is a perspective view of a patient specific outsole according to an embodiment of the present disclosure.

FIG. 17E is a bottom view of the patient specific outsole of FIG. 17D.

FIG. 17F is a side view of the patient specific outsole of FIG. 17D.

FIG. 17G is a perspective view of a rocker outsole according to an embodiment of the present disclosure.

FIG. 17H is a bottom view of the rocker outsole of FIG. 17G.

FIG. 17J is a side view of the rocker outsole of FIG. 17G.

FIG. 18A is a perspective view of a baseline fixture according to an embodiment of the present disclosure.

FIG. 18B is a perspective view of a base plate according to an embodiment of the present disclosure.

FIG. 19A is a perspective view of a baseline fixture according to an embodiment of the present disclosure.

FIG. 19B is a top view of the baseline fixture of FIG. 19A.

FIG. 19C is a side section view of the baseline fixture of FIG. 19B.

FIG. 20 is a perspective view of an adjustment platform according to an embodiment of the present disclosure.

FIG. 21 is a perspective view of a locating bridge according to an embodiment of the present disclosure.

FIG. 22 is a perspective view of an adjustment platform according to an embodiment of the present disclosure.

FIG. 23 is a perspective view of an adjustment platform according to an embodiment of the present disclosure.

FIG. 24A is a perspective view of an exemplary foot on the baseline fixture of FIG. 19A according to an embodiment of the present disclosure.

FIG. 24B is a top view of the exemplary foot on the baseline fixture of FIG. 24A.

FIG. 25 is a perspective view of a gait modifier outsole and an adjustment platform according to an embodiment of the present disclosure.

FIG. 26 is a perspective view of the gait modifier outsole and the adjustment platform of FIG. 25 and a baseline fixture according to an embodiment of the present disclosure.

FIG. 27 is a perspective view of the gait modifier outsole and the adjustment platform of FIG. 25.

FIG. 28 is a perspective view of the gait modifier outsole and the adjustment platform of FIG. 25, a locating bridge, and a plate according to an embodiment of the present disclosure.

FIG. 29 is a perspective view of the gait modifier outsole, the adjustment platform, the locating bridge, and the plate of FIG. 28.

FIG. 30 is an exemplary method for adjusting a gait modifier based on anatomic features according to an embodiment of the present disclosure.

FIG. 31 is an exemplary method for adjusting a gait modifier based on a gait analysis according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to gait modification systems and methods. Those skilled in the art will recognize that the following description is merely illustrative of the principles of the technology, which may be applied in various ways to provide many alternative embodiments. For the purposes of illustrating the concepts of the present design, the present disclosure describes systems and methods for adjustable gait modifier shoe outsoles. However, it will be understood that other variations and uses are contemplated.

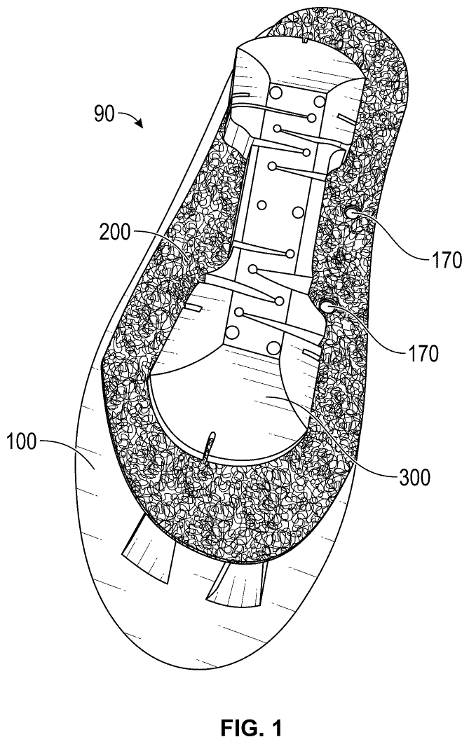

FIG. 1 is a bottom perspective view of a gait modifier system 90 according to an embodiment of the present disclosure. Modifying an outsole of a shoe may significantly impact gait mechanics, reduce stress on joints, minimize joint pain, and/or minimize joint discomfort. A modified outsole may offload pressure from specific joints, particularly an ankle joint and/or a knee joint, which may alleviate pain and/or discomfort in those joints.

By wearing a gait modifier shoe 100 while ambulating and defining a point of contact and/or a plane of contact between a foot and the ground, that may be different than an existing point of contact and/or plane of contact between the foot and the ground, the orientation of the foot during ambulation may be altered, the balance of the patient during ambulation may be altered, and/or a force propagating through a bone and/or a joint of the patient may be altered. The gait modifier shoe 100 may be configured so that the orientation, the balance, and/or the force propagation may be altered in a way to improve gait mechanics, reduce pain, reduce discomfort, improve balance, and/or improve a stride of the patient.

Additionally, or alternatively, a modified outsole may redistribute pressure across a joint by altering a heel-to-toe drop portion of a gait. Additionally, or alternatively, a modified outsole may include a modified stiffness and/or rigidity in order to reduce excessive pronation and/or inhibit overcompensation in joints. Additionally, or alternatively, a modified outsole may ensure even weight distribution which may minimize abnormal pressure points, thereby reducing joint stress. A modified outsole may minimize abnormal stress on joints, correct biomechanical issues, prevent further pain, improve alignment, reduce wear on joints, and/or optimize gait efficiency thereby reducing a risk of joint pain, for example, in the knees, hips, and/or lower back.

The gait modifier system 90 may be configurable and/or adjustable based on analysis of gait mechanics of a patient. Additionally, or alternatively, the gait modifier system 90 may be configurable and/or adjustable based on measurements of one or more anatomic features of the patient. The gait modifier system 90 may include a gait modifier outsole 300 that may be configurable and/or adjustable in an anterior, a posterior, and/or a rotational direction from heel strike to toe off of a left foot and/or a right foot to optimize a patient's gait cycle. Additionally, or alternatively, the gait modifier system 90 may be configured and/or adjusted to train neuroplasticity of a brain of the patient to remember a customized and/or optimized gait.

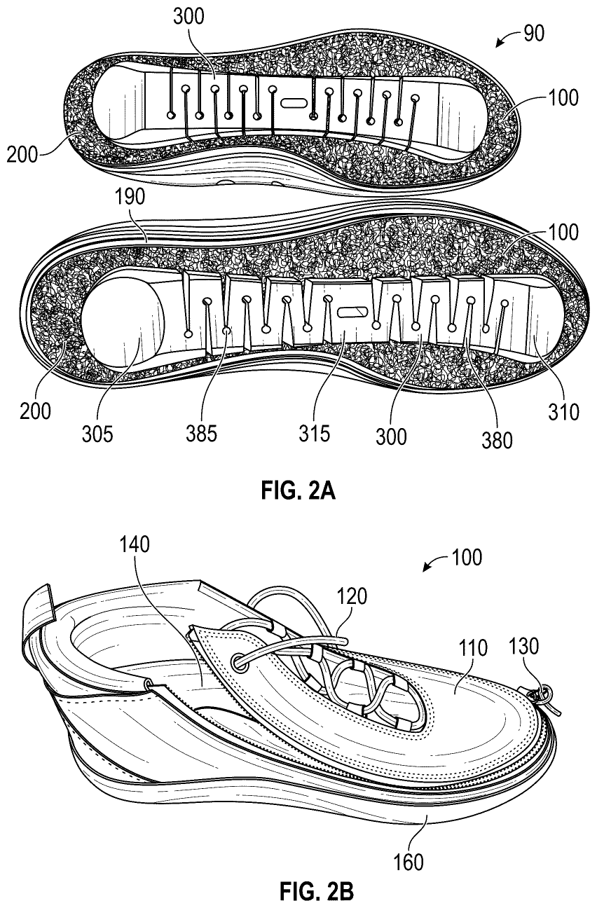

FIG. 2A is a bottom view of a first gait modifier system 90 configured for a first patient and a second gait modifier system 90 configured for a second patient according to an embodiment of the present disclosure. The gait modifier system 90 may include a gait modifier shoe 100, a plate 200, and a gait modifier outsole 300. FIG. 2B is a perspective view of the gait modifier shoe 100 according to an embodiment of the present disclosure.

The gait modifier outsole 300 may include a hindfoot portion 305, a forefoot portion 310, and a central portion 315 connecting the hindfoot portion 305 and the forefoot portion 310. The gait modifier shoe 100 may include a shoe outsole 160. The gait modifier outsole 300 may be configured to attach to the shoe outsole 160. The hindfoot portion 305 may be adjustably positionable relative to the shoe outsole 160 independently of the forefoot portion 310. The forefoot portion 310 may be adjustably positionable relative to the shoe outsole 160 independently of the hindfoot portion 305.

Additionally, or alternatively, the hindfoot portion 305 may be positionable at a first position and orientation relative to the plate 200 and the forefoot portion 310 may be positionable at a second position and orientation relative to the plate 200. The first position and the second position may be adjustable with the gait modifier outsole 300 detached from the plate 200. Further, the first position and the second position may not be adjustable with the gait modifier outsole 300 attached to the plate 200.

The gait modifier shoe 100 may include a shoe upper 110, a shoe outsole 160, and one or more locating features 170. The gait modifier shoe 100 may be configured as an athletic shoe, a dress shoe, a casual shoe, a boot, a sandal, a heeled shoe, a flat shoe, a slip-on shoe, a loafer, and/or other type of footwear configured to be worn on a foot. The shoe upper 110 may be configured to, at least partially, envelope a foot to retain the gait modifier shoe 100 on the foot.

The shoe upper 110 may include a first fastener 120, a second fastener 130, a tongue 140. The first fastener 120 and the second fastener 130 may allow the shoe upper 110 to open to ease insertion of the foot into the gait modifier shoe 100. The first fastener 120 may be configured as laces that may be loosened to ease insertion of the foot into the gait modifier shoe 100 and tightened to retain the foot within the gait modifier shoe 100. The second fastener 130 may be configured as a zipper that may be opened to ease insertion of the foot into the gait modifier shoe 100 and closed to retain the foot within the gait modifier shoe 100.

Alternatively, the gait modifier shoe 100 may include only the first fastener 120 and not the second fastener 130. Additionally, or alternatively, the first fastener 120 and/or the second fastener 130 may be configured as a buckle, a strap, a hook-and-loop type closure (ex: Velcro®), an elastic type cinching closure, one or more snaps, one or more buttons, an elastic gore, and/or other closure mechanism configured to retain the foot within the gait modifier shoe 100.

The shoe outsole 160 may be configured to receive the plate 200 and/or the gait modifier outsole 300 so that, with a patient standing and wearing the gait modifier shoe 100, the gait modifier outsole 300 may be positioned between the shoe outsole 160 and the ground. Additionally, the shoe outsole 160 and the gait modifier outsole 300 may be configured so that, with the patient wearing the gait modifier shoe 100 and ambulating, the shoe outsole 160 does not contact the ground and the gait modifier outsole 300 does contact the ground. The one or more locating features 170 may be configured to facilitate alignment and/or positioning of the plate 200 relative to the shoe outsole 160.

The shoe outsole 160 may include a fastening surface 190 configured to attach the plate 200 to the shoe outsole 160. The fastening surface 190 may include a hook-and-loop fastener type surface, (ex: Velcro®). Additionally, or alternatively, the fastening surface 190 may include other means for removably attaching the plate 200 to the shoe outsole 160, such as: magnets, removeable adhesive, clevis pins, cotter pins, linchpins, retaining rings, threaded fasteners, snaps, turn-button fasteners, clips, and/or another removably and securably attachable fastening mechanism. The plate 200 may include a fastening feature that is complementary to the fastening surface 190 so that the plate 200 may be removably and securably attachable to the shoe outsole 160. The fastening surface 190 may be configured so that the gait modifier outsole 300 may be loosely attached to the plate 200, and adjustable relative to the plate, prior to final tightening and securing of the gait modifier outsole 300 to the plate 200. Additionally, or alternatively, the fastening surface may be configured so that, after securely attaching the gait modifier outsole 300 to the plate 200, the fastening surface may be loosened so that the gait modifier outsole 300 remains captive but adjustable relative to the plate.

The plate 200 may be configured to attach to the shoe outsole 160. The gait modifier outsole 300 may be configured to attach to the plate 200. The gait modifier outsole 300 may be attached to the plate 200 prior to the plate being attached to the shoe outsole 160.

Alternatively, the gait modifier system 90 may include a gait modifier shoe 100 and a gait modifier outsole 300. The gait modifier outsole 300 may removably attach directly to the shoe outsole 160 of the gait modifier shoe 100, without the use of the plate 200. The gait modifier outsole 300 may be removably positionable on the shoe outsole 160.

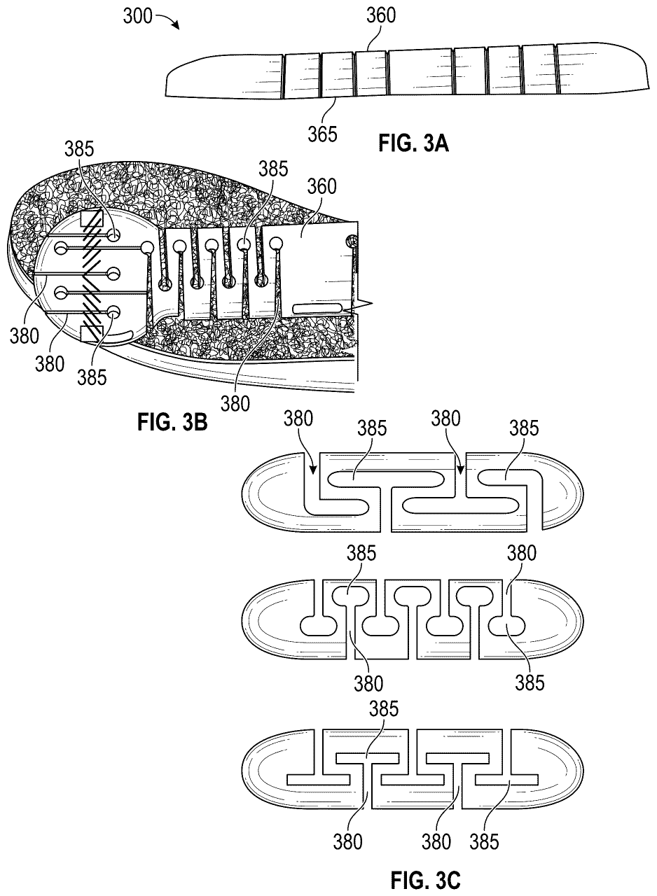

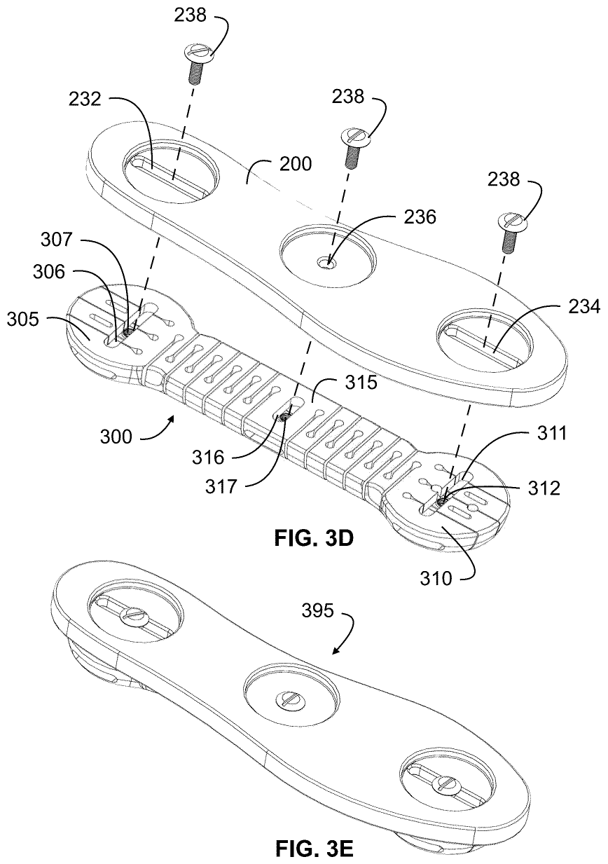

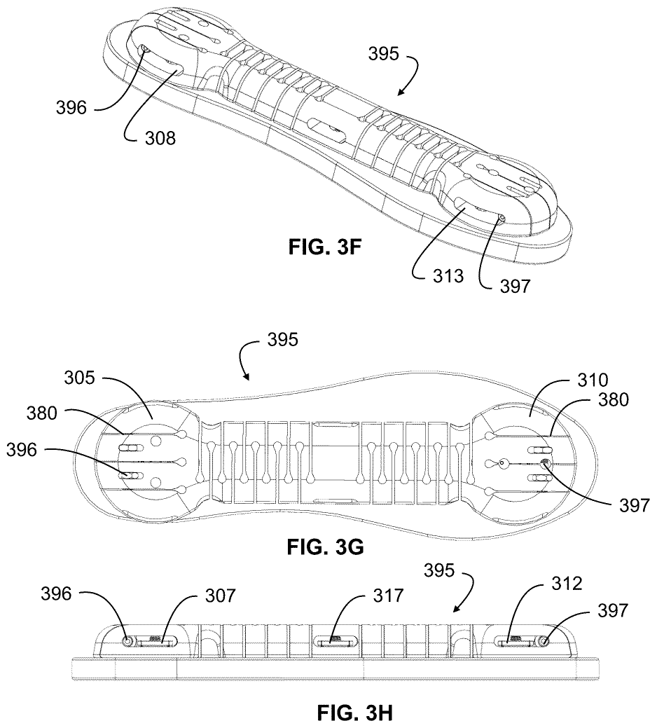

FIG. 3A is a side view of a gait modifier outsole 300 according to an embodiment of the present disclosure. The gait modifier outsole 300 may include a ground contacting surface 360 and a shoe facing surface 365. The shoe facing surface 365 may be configured to be removably and securably attachable to the plate 200. Alternatively, the shoe facing surface 365 may be configured to be removably and securably attachable to the shoe outsole 160.

The ground contacting surface 360 may be configured so that the hindfoot portion 305, the forefoot portion 310, and the central portion 315 contact the ground during ambulation. Alternatively, the ground contacting surface 360 may be configured so that the central portion 315 does not contact the ground during ambulation. The central portion 315 may be configured as a generally planar surface.

The shoe facing surface 365 may include a hook-and-loop fastener type surface, (ex: Velcro®). Additionally, or alternatively, the shoe facing surface 365 may include other means for removably attaching the gait modifier outsole 300 to the plate 200, such as: magnets, removeable adhesive, clevis pins, cotter pins, linchpins, retaining rings, threaded fasteners, snaps, turn-button fasteners, clips, and/or another removably and securably attachable fastening mechanism.

The plate 200 may include a fastening feature that is complementary to the shoe facing surface 365 so that the shoe facing surface 365 may be removably and securably attachable to the plate 200. Alternatively, the shoe outsole 160 may include a fastening feature that is complementary to the shoe facing surface 365 so that the shoe facing surface 365 may be removably and securably attachable to the shoe outsole 160.

With the gait modifier outsole 300 securably attached to the gait modifier shoe 100, and with the patient wearing the gait modifier shoe 100, the gait modifier outsole 300 may be configured to modify the gait mechanics during ambulation of the patient.

FIG. 3B. is a perspective view of a gait modifier outsole 300 according to an embodiment of the present disclosure. The gait modifier outsole 300 may include a plurality of slots 380. The plurality of slots 380 may be configured to facilitate portions of the gait modifier outsole 300 to be expanded, contracted, rotated, and/or angled into a position while maintaining a relatively planar ground contacting surface 360.

A longitudinal axis of each of the plurality of slots 380 may be generally perpendicular to a longitudinal axis of the gait modifier outsole 300. Additionally, or alternatively, the longitudinal axis of each of the plurality of slots 380 may be generally parallel to the longitudinal axis of the gait modifier outsole 300. Additionally, or alternatively, the longitudinal axis of each of the plurality of slots 380 may form an acute angle and/or an obtuse angle relative to the longitudinal axis of the gait modifier outsole 300. The gait modifier outsole 300 may include a plurality of slots 380 having multiple different orientations relative to a longitudinal axis of the gait modifier outsole 300.

Each of the plurality of slots 380 may include a stress relief aperture 385 configured to minimize internal material stresses proximate the slots 380 during expansion, contraction, rotation, and/or angulation of the gait modifier outsole 300. The stress relief apertures 385 may have a generally arcuate profile such that the arcuate profile extends beyond a width of the slots 380.

FIG. 3C is a sketch of alternate slot patterns of a gait modifier outsole 300 according to an embodiment of the present disclosure. The gait modifier outsole 300 may include a plurality of slots 380 and a corresponding plurality of stress relief apertures 385 having a shape other than previously described. For example, the plurality of slots 380 and the corresponding plurality of stress relief apertures 385 may have an ‘L’ shape, a lollipop shape, a ‘T’ shape, and/or other shape that may facilitate portions of the gait modifier outsole 300 being expanded, contracted, rotated, and/or angled while minimizing internal material stresses proximate the slots 380.

FIG. 3D is an exploded perspective view of a gait modifier 395 according to an embodiment of the present disclosure. FIG. 3E is a perspective view of the gait modifier 395. The hindfoot portion 305 may include a hindfoot aperture 306 and a hindfoot fastener receiver 307. The hindfoot fastener receiver 307 may be configured to receive a fastener 238.

The forefoot portion 310 may include a forefoot aperture 311 and a forefoot fastener receiver 312. The forefoot fastener receiver 312 may be configured to receiver a fastener 238. The central portion 315 may include a central aperture 316 and a central fastener receiver 317. The central fastener receiver 317 may be configured to receive a fastener 238.

A plate 200 may include a first aperture 232, a second aperture 234, and a third aperture 236. The first aperture 232 may be at least partially aligned with the hindfoot aperture 306. The first aperture 232 may be sized so that a fastener 238 may at least partially pass through the first aperture 232 to secure the plate 200 to the gait modifier outsole 300. The first aperture 232 may be configured as an elongated slot so that the fastener 238 may at least partially pass through at a plurality locations along the first aperture 232.

The hindfoot aperture 306 may be configured as an elongated slot so that the fastener 238 may at least partially pass through at a plurality locations along the hindfoot aperture 306. The hindfoot aperture 306 may be generally perpendicular to the first aperture 232. The hindfoot fastener receiver 307 may be configured to receive the fastener 238 after the fastener 238 has passed through the first aperture 232 and the hindfoot aperture 306. Additionally, the hindfoot fastener receiver 307 may be moveable relative to the hindfoot aperture 306 so that the fastener 238 may be received by the hindfoot fastener receiver 307 with the hindfoot portion 305 adjusted from a first position to a second position.

The second aperture 234 may be at least partially aligned with the forefoot aperture 311. The second aperture 234 may be sized so that a fastener 238 may at least partially pass through second aperture 234 to secure the plate 200 to the gait modifier outsole 300. The second aperture 234 may be configured as an elongated slot so that the fastener 238 may at least partially pass through at a plurality locations along the second aperture 234.

The forefoot aperture 311 may be configured as an elongated slot so that the fastener 238 may at least partially pass through at a plurality locations along the forefoot aperture 311. The forefoot aperture 311 may be generally perpendicular to the second aperture 234. The forefoot fastener receiver 312 may be configured to receive the fastener 238 after the fastener 238 has passed through the second aperture 234 and the forefoot aperture 311. Additionally, the forefoot fastener receiver 312 may be moveable relative to forefoot aperture 311 so that the fastener 238 may be received by the forefoot fastener receiver 312 with the forefoot portion 310 adjusted from a third position to a fourth position.

The third aperture 236 may be at least partially aligned with the central aperture 316. The third aperture 236 may be sized so that a fastener 238 may at least partially pass through second aperture 234 to secure the plate 200 to the gait modifier outsole 300.

The central aperture 316 may be configured as an elongated slot so that the fastener 238 may at least partially pass through at a plurality locations along the central aperture 316. The central aperture 316 may be generally perpendicular a longitudinal axis of the gait modifier outsole 300. The central fastener receiver 317 may be configured to receive the fastener 238 after the fastener 238 has passed through the third aperture 236 and the central aperture 316. Additionally, the central fastener receiver 317 may be moveable relative to central aperture 316 so that the fastener 238 may be received by the central fastener receiver 317 with the central portion 315 adjusted from a fifth position to a sixth position.

FIG. 3F is a perspective view of the gait modifier 395, FIG. 3G is a bottom view of the gait modifier 395, and FIG. 3H is a side view of the gait modifier 395. The plurality of slots 380 may be located within the hindfoot portion 305. The hindfoot portion 305 may include a first lateral aperture 308 and a first turnbuckle 396 located within the first lateral aperture 308. The first turnbuckle 396 may be configured to modify a width of the slots 380 when actuated, thereby expanding, contracting, rotating, and/or angling the hindfoot portion 305 relative to an initial position.

Additionally, or alternatively, the plurality of slots 380 may be located within the forefoot portion 310. The forefoot portion 310 may include a second lateral aperture 313 and a second turnbuckle 397 located within the second lateral aperture 313. The second turnbuckle 397 may be configured to modify a width of the slots 380 when actuated, thereby expanding, contracting, rotating, and/or angling the hindfoot portion 305 relative to an initial position. An expanded hindfoot portion 305 and/or an expanded forefoot portion 310 may provide an increased ground contacting surface 360 and, therefore, increased stability for the patient during ambulation.

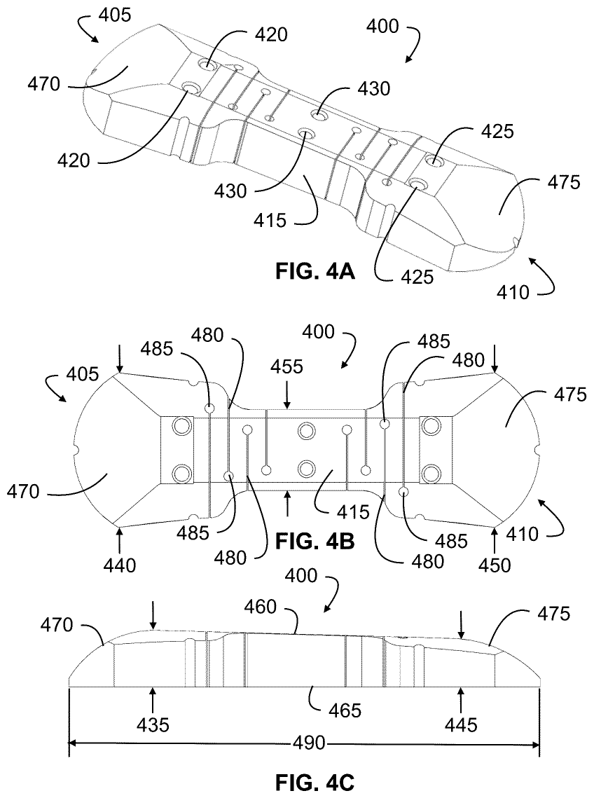

FIG. 4A is a perspective view of a small transition directional gait modifier outsole 400 according to an embodiment of the present disclosure. FIG. 4B is a bottom view of the small transition directional gait modifier outsole 400 and FIG. 4C is a side view of the small transition directional gait modifier outsole 400. The gait modifier outsole 300 may be configured as the small transition directional gait modifier outsole 400. The small transition directional gait modifier outsole 400 may include similar functions and features previously described for the gait modifier outsole 300. Conversely, the gait modifier outsole 300 may include similar functions and features as the small transition directional gait modifier outsole 400 described below.

The various features of the small transition directional gait modifier outsole 400 with similar reference numbers as those of the gait modifier outsole 300 may be similar in function and/or configuration to their counterparts of the gait modifier outsole 300. Differences between the gait modifier outsole 300 and the small transition directional gait modifier outsole 400 will be set forth below.

The small transition directional gait modifier outsole 400 may be configured to be adjusted (expanded, contracted, rotated, and/or angled) based on a patient's anatomy and/or a patient's gait mechanics. The small transition directional gait modifier outsole 400 may then be securably attached to the plate 200 and/or the shoe outsole 160 to fix the adjustments relative to the gait modifier shoe 100.

The small transition directional gait modifier outsole 400 may include a hindfoot portion 405, a forefoot portion 410 and a central portion 415 connecting the hindfoot portion 405 and the forefoot portion 410. The small transition directional gait modifier outsole 400 may further include a ground contacting surface 460, a shoe facing surface 465, and an outsole length 490. The ground contacting surface 460 may be configured as a generally planar ground contacting surface. The outsole length 490 may be a maximum distance between the hindfoot portion 405 and forefoot portion 410. The outsole length 490 may be sized to be used in conjunction with a gait modifier shoe 100 sized between size 5 and size 9.5.

The hindfoot portion 405 may include one or more hindfoot locating apertures 420, a hindfoot height 435, a hindfoot width 440, and a heel touch surface 470. The heel touch surface 470 may be located on the hindfoot portion 405 and may extend, at least partially, between the ground contacting surface 460 and the shoe facing surface 465. The heel touch surface 470 may be configured as a continuous two-dimensional surface, such as a radius surface, a flat surface, and/or a combination of one or more radius surfaces and/or one or more flat surfaces. In an embodiment, the heel touch surface 470 may be configured as a two-dimensional radius that is tangent to the ground contacting surface 460 and may extend towards the shoe facing surface 465.

The two-dimensional surface may be configured as a unidirectional surface. The unidirectional surface may facilitate control of the heel strike and/or toe off motion during a gait cycle. The unidirectional surface may guide the patient's foot so that the unidirectional surface is ground contacting during a heel strike and/or toe off of a gait cycle.

Additionally, or alternatively, the unidirectional surface may be configured to orient a patient's foot so that the unidirectional surface is ground contacting during the gait cycle. The unidirectional surface may inhibit rotation of the gait modifier outsole 300 normal to the unidirectional surface during ambulation.

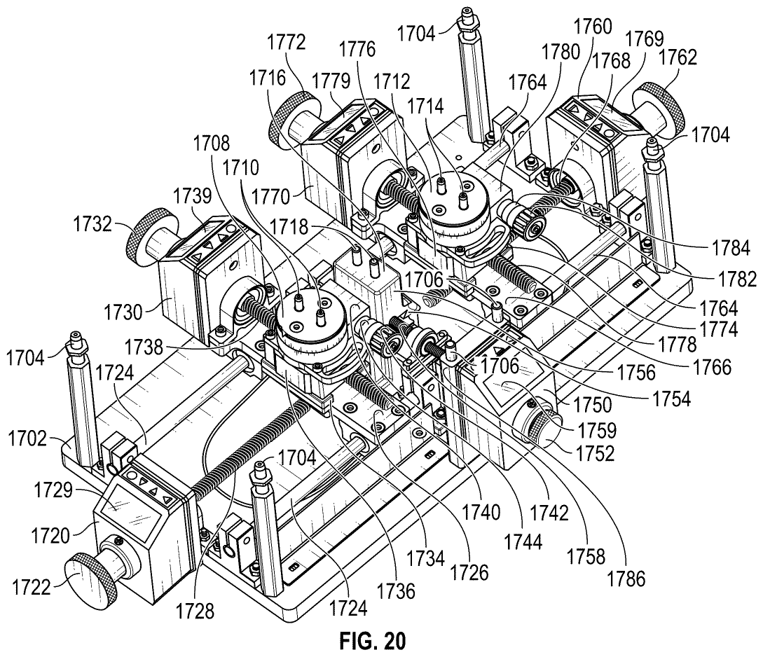

The one or more hindfoot locating apertures 420 may be configured to receive one or more first outsole locating features 1710 (as shown in FIG. 20) to facilitate adjustment and/or positioning of the hindfoot portion 405. The hindfoot portion 405 may be adjustably positionable independent of the forefoot portion 410.

The hindfoot height 435 may be the maximum distance between the ground contacting surface 460 and the shoe facing surface 465 in the hindfoot portion 405. The hindfoot width 440 may be the maximum width of the hindfoot portion 405 measured in a plane that is parallel to the shoe facing surface 465.

The forefoot portion 410 may include one or more forefoot locating apertures 425, a forefoot height 445, a forefoot width 450, and a toe off surface 475. The toe off surface 475 may be located on the forefoot portion 410 and may extend, at least partially, between the ground contacting surface 460 and the shoe facing surface 465. The toe off surface 475 may be configured as a continuous two-dimensional surface, such as a radius surface, a flat surface, and/or a combination of one or more radius surfaces and/or one or more flat surfaces. In an embodiment, the toe off surface 475 may be configured as a two-dimensional radius that is tangent to the ground contacting surface 460 and may extend towards the shoe facing surface 465.

The one or more forefoot locating apertures 425 may be configured to receive one or more second outsole locating features 1714 (as shown in FIG. 20) to facilitate adjustment and/or positioning of the forefoot portion 410. The forefoot portion 410 may be adjustably positionable independent of the hindfoot portion 405.

The forefoot height 445 may be the maximum distance between the ground contacting surface 460 and the shoe facing surface 465 in the forefoot portion 410. The forefoot width 450 may be the maximum width of the forefoot portion 410 measured in a plane that is parallel to the shoe facing surface 465.

The ground contacting surface 460 may be generally parallel to the shoe facing surface 465. Alternatively, the ground contacting surface 460 may be angled along an anterior-posterior axis. The ground contacting surface 460 may be inverted so that a distance between the ground contacting surface 460 and the shoe facing surface 465 is be less along a medial portion of the small transition directional gait modifier outsole 400 relative to a lateral portion of the small transition directional gait modifier outsole 400. Alternatively, the ground contacting surface 460 may be everted so that a distance between the ground contacting surface 460 and the shoe facing surface 465 is be greater along a medial portion of the small transition directional gait modifier outsole 400 relative to a lateral portion of the small transition directional gait modifier outsole 400.

Alternatively, the ground contacting surface 460 may have a generally arcuate shape having a center point. The generally arcuate shape may include a single radius, a spline, and/or a polyline. The center point may be centered relative to a sagittal plane and a coronal plane. Alternatively, the center point may be located anteriorly or posteriorly relative to the coronal plane. Additionally, or alternatively, the center point may be located medially or laterally relative to the sagittal plane.

The central portion 415 may include one or more central locating apertures 430 and a central width 455. The one or more central locating apertures 430 may be configured to receive one or more third outsole locating features 1718 (as shown in FIG. 20) to facilitate adjustment and/or positioning of the central portion 415. The central portion 415 may be positioned and/or adjusted independently of the hindfoot portion 405 and/or the forefoot portion 410.

The small transition directional gait modifier outsole 400 may include a plurality of slots 480 and a corresponding plurality of stress relief apertures 485. The plurality of slots 480 may be located in the hindfoot portion 405, the forefoot portion 410, and/or the central portion 415. The hindfoot portion 405 may be configured to cooperate with the forefoot portion 410 during ambulation of the patient while wearing the gait modifier shoe 100 to modify the gait mechanics of the patient.

The small transition directional gait modifier outsole 400 may be configured so that the hindfoot height 435 is greater than the forefoot height 445. The small transition directional gait modifier outsole 400 may further be configured so that the hindfoot width 440 and the forefoot width 450 are generally equal. The small transition directional gait modifier outsole 400 may further be configured so that the hindfoot width 440 and the forefoot width 450 are greater than the central width 455. The small transition directional gait modifier outsole 400 may further be configured so that heel touch surface 470 is generally congruent to the toe off surface 475.

The hindfoot height 435 may be adjustable through a threaded mechanism and/or by securing modular components to the hindfoot portion 405. The forefoot height 445 may be adjustable through a threaded mechanism and/or by securing modular components to the hindfoot portion 405. The hindfoot height 435 may be adjustable independently of the forefoot height 445. The forefoot height 445 may be adjustable independently of the hindfoot height 435.

The small transition directional gait modifier outsole 400 has been described herein as including a hindfoot portion 405 and a forefoot portion 410. However, based on patient anatomy and/or patient gait mechanics, it may be beneficial to reverse the orientation of the small transition directional gait modifier outsole 400 relative to the gait modifier shoe 100, so that the hindfoot portion 405 is located proximate a forefoot of the patient and the forefoot portion 410 is located proximate a hindfoot of the patient.

In an embodiment, the small transition directional gait modifier outsole 400 may be one of a set of differently sized gait modifier outsoles 300 each having a different hindfoot height 435, a different forefoot height 445, a different hindfoot width 440, a different forefoot width 450, a different central width 455, a different heel touch surface 470, a different toe off surface 475, and/or a different outsole length 490.

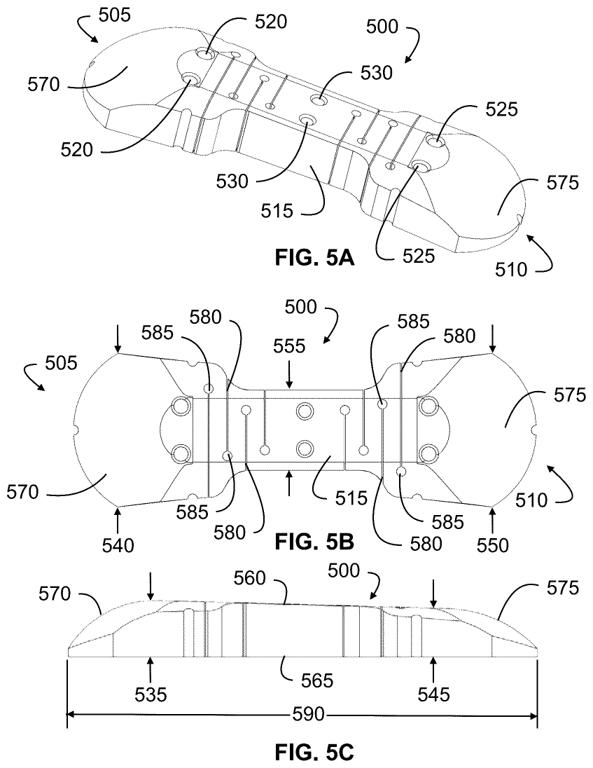

FIG. 5A is a perspective view of a small transition omni gait modifier outsole 500 according to an embodiment of the present disclosure. FIG. 5B is a bottom view of the small transition omni gait modifier outsole 500 and FIG. 5C is a side view of the small transition omni gait modifier outsole 500. The small transition omni gait modifier outsole 500 may include similar functions and features previously described for the gait modifier outsole 300. Conversely, the gait modifier outsole 300 may include similar functions and features as the small transition omni gait modifier outsole 500 described below.

The various features of the small transition omni gait modifier outsole 500 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the small transition omni gait modifier outsole 500 will be set forth below.

The small transition omni gait modifier outsole 500 may include a hindfoot portion 505, a forefoot portion 510, and a central portion 515 connecting the hindfoot portion 505 and the forefoot portion 510. The small transition omni gait modifier outsole 500 may further include a ground contacting surface 560, a shoe facing surface 565, and an outsole length 590. The outsole length 590 may be sized to be used in conjunction with a gait modifier shoe 100 sized between size 5 and size 9.5.

The hindfoot portion 505 may include one or more hindfoot locating apertures 520, a hindfoot height 535, a hindfoot width 540, and a heel touch surface 570. The heel touch surface 570 may be located on the hindfoot portion 505 and may extend, at least partially, between the ground contacting surface 560 and the shoe facing surface 565. The heel touch surface 570 may be configured as a continuous three-dimensional surface, such as a spherical radius surface, a cylindrical surface, and/or a combination of one or more spherical radius surfaces and/or one or more cylindrical surfaces. In an embodiment, the heel touch surface 570 may be configured as a three-dimensional spherical radius that is tangent to the ground contacting surface 560 and may extend towards the shoe facing surface 565. The three-dimensional surface may be configured as an omnidirectional surface. The omnidirectional surface may allow rotation of the gait modifier outsole about a longitudinal axis of the gait modifier outsole 300 during ambulation.

The forefoot portion 510 may include one or more forefoot locating apertures 525, a forefoot height 545, a forefoot width 550, and a toe off surface 575. The toe off surface 575 may be configured as a continuous three-dimensional surface, such as a spherical radius surface, a cylindrical surface, and/or a combination of one or more spherical radius surfaces and/or one or more cylindrical surfaces. In an embodiment, the toe off surface 575 may be configured as a three-dimensional spherical radius that is tangent to the ground contacting surface 560 and may extend towards the shoe facing surface 565.

The central portion 515 may include one or more central locating apertures 530 and a central width 555. The small transition omni gait modifier outsole 500 may include a plurality of slots 580 and a corresponding plurality of stress relief apertures 585.

The small transition omni gait modifier outsole 500 may be configured so that the hindfoot height 535 is greater than the forefoot height 545. The small transition omni gait modifier outsole 500 may further be configured so that the hindfoot width 540 and the forefoot width 550 are generally equal. The small transition omni gait modifier outsole 500 may further be configured so that the hindfoot width 540 and the forefoot width 550 are greater than the central width 555. The small transition omni gait modifier outsole 500 may further be configured so that heel touch surface 570 is generally congruent to the toe off surface 575.

In an embodiment, the small transition omni gait modifier outsole 500 may be one of a set of differently sized gait modifier outsoles 300 each having a different hindfoot height 535, a different forefoot height 545, a different hindfoot width 540, a different forefoot width 550, a different central width 555, a different heel touch surface 570, a different toe off surface 575, and/or a different outsole length 590.

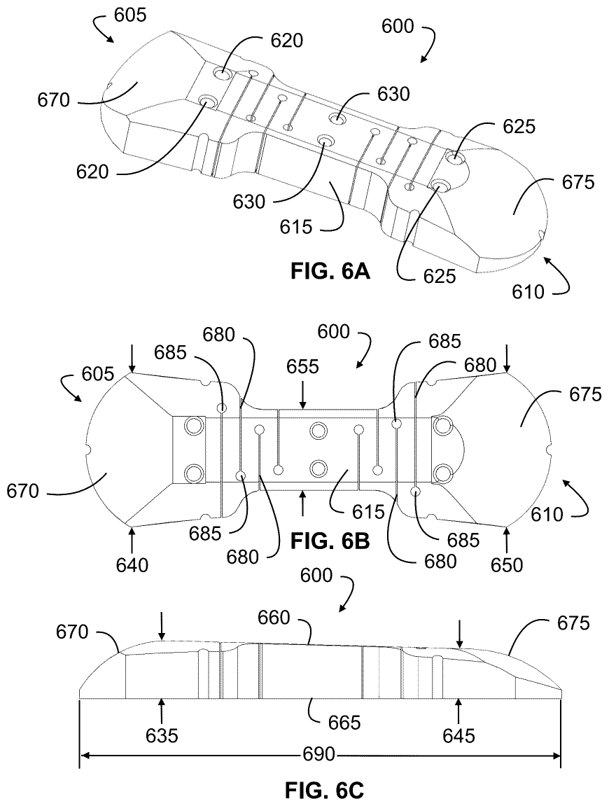

FIG. 6A is a perspective view of a small transition hybrid gait modifier outsole 600 according to an embodiment of the present disclosure. FIG. 6B is a bottom view of the small transition hybrid gait modifier outsole 600 and FIG. 6C is a side view of the small transition hybrid gait modifier outsole 600. The small transition hybrid gait modifier outsole 600 may include similar functions and features previously described for the gait modifier outsole 300. Conversely, the gait modifier outsole 300 may include similar functions and features as the small transition hybrid gait modifier outsole 600 described below.

The various features of the small transition hybrid gait modifier outsole 600 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the small transition hybrid gait modifier outsole 600 will be set forth below.

The small transition hybrid gait modifier outsole 600 may include a hindfoot portion 605, a forefoot portion 610, and a central portion 615 connecting the hindfoot portion 605 and the forefoot portion 610. The small transition hybrid gait modifier outsole 600 may further include a ground contacting surface 660, a shoe facing surface 665, and an outsole length 690. The outsole length 690 may be sized to be used in conjunction with a gait modifier shoe 100 sized between size 5 and size 9.5.

The hindfoot portion 605 may include one or more hindfoot locating apertures 620, a hindfoot height 635, a hindfoot width 640, and a heel touch surface 670. The heel touch surface 670 may be located on the hindfoot portion 605 and may extend, at least partially, between the ground contacting surface 660 and the shoe facing surface 665. The heel touch surface 670 may be configured as a continuous two-dimensional surface, such as a radius surface, a flat surface, and/or a combination of one or more radius surfaces and/or one or more flat surfaces. In an embodiment, the heel touch surface 670 may be configured as a two-dimensional radius that is tangent to the ground contacting surface 660 and may extend towards the shoe facing surface 665.

The forefoot portion 610 may include one or more forefoot locating apertures 625, a forefoot height 645, a forefoot width 650, and a toe off surface 675. The toe off surface 675 may be configured as a continuous three-dimensional surface, such as a spherical radius surface, a cylindrical surface, and/or a combination of one or more spherical radius surfaces and/or one or more cylindrical surfaces. In an embodiment, the toe off surface 675 may be configured as a three-dimensional spherical radius that is tangent to the ground contacting surface 660 and may extend towards the shoe facing surface 665. The two-dimensional surface may be configured as a unidirectional surface. The three-dimensional surface may be configured as an omnidirectional surface. The unidirectional surface may facilitate more effective control of the heel strike and/or toe off motion during a gait cycle than the omnidirectional surface.

The central portion 615 may include one or more central locating apertures 630 and a central width 655. The small transition hybrid gait modifier outsole 600 may include a plurality of slots 680 and a corresponding plurality of stress relief apertures 685.

The small transition hybrid gait modifier outsole 600 may be configured so that the hindfoot height 635 is greater than the forefoot height 645. The small transition hybrid gait modifier outsole 600 may further be configured so that the hindfoot width 640 and the forefoot width 650 are generally equal. The small transition hybrid gait modifier outsole 600 may further be configured so that the hindfoot width 640 and the forefoot width 650 are greater than the central width 655. The small transition hybrid gait modifier outsole 600 may further be configured so that heel touch surface 670 is configured as a two-dimension surface and the toe off surface 675 is configured as a three-dimensional surface.

In an embodiment, the small transition hybrid gait modifier outsole 600 may be one of a set of differently sized gait modifier outsoles 300 each having a different hindfoot height 635, a different forefoot height 645, a different hindfoot width 640, a different forefoot width 650, a different central width 655, a different heel touch surface 670, a different toe off surface 675, and/or a different outsole length 690.

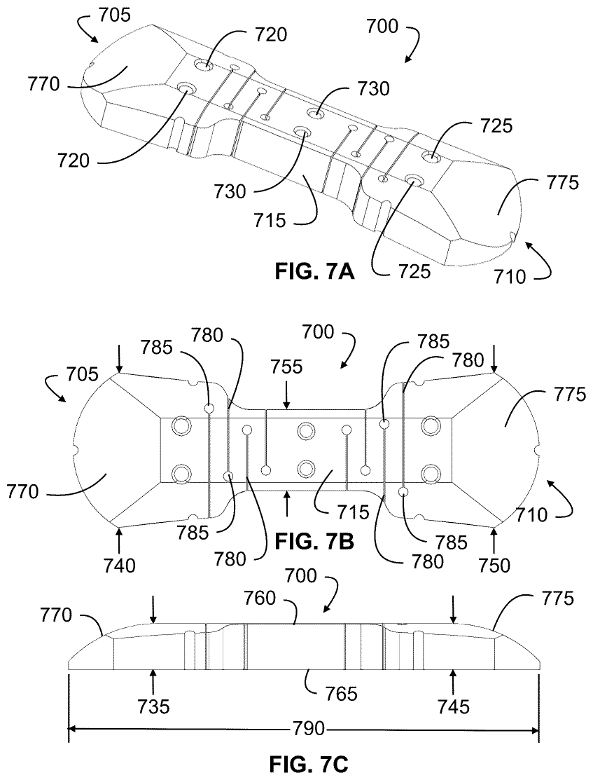

FIG. 7A is a perspective view of a small flat directional gait modifier outsole 700 according to an embodiment of the present disclosure. FIG. 7B is a bottom view of the small flat directional gait modifier outsole 700 and FIG. 7C is a side view of the small flat directional gait modifier outsole 700. The small flat directional gait modifier outsole 700 may include similar functions and features previously described for the gait modifier outsole 300. Conversely, the gait modifier outsole 300 may include similar functions and features as the small flat directional gait modifier outsole 700 described below.

The various features of the small flat directional gait modifier outsole 700 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the small flat directional gait modifier outsole 700 will be set forth below.

The small flat directional gait modifier outsole 700 may include a hindfoot portion 705, a forefoot portion 710, and a central portion 715 connecting the hindfoot portion 705 and the forefoot portion 710. The small flat directional gait modifier outsole 700 may further include a ground contacting surface 760, a shoe facing surface 765, and an outsole length 790. The outsole length 790 may be sized to be used in conjunction with a gait modifier shoe 100 sized between size 5 and size 9.5.

The hindfoot portion 705 may include one or more hindfoot locating apertures 720, a hindfoot height 735, a hindfoot width 740, and a heel touch surface 770. The heel touch surface 770 may be located on the hindfoot portion 705 and may extend, at least partially, between the ground contacting surface 760 and the shoe facing surface 765. The heel touch surface 770 may be configured as a continuous two-dimensional surface, such as a radius surface, a flat surface, and/or a combination of one or more radius surfaces and/or one or more flat surfaces. In an embodiment, the heel touch surface 770 may be configured as a two-dimensional radius that is tangent to the ground contacting surface 760 and may extend towards the shoe facing surface 765. The two-dimensional surface may be configured as a unidirectional surface. The unidirectional surface may facilitate control of the heel strike and/or toe off motion during a gait cycle.

The forefoot portion 710 may include one or more forefoot locating apertures 725, a forefoot height 745, a forefoot width 750, and a toe off surface 775. The toe off surface 775 may be located on the forefoot portion 710 and may extend, at least partially, between the ground contacting surface 760 and the shoe facing surface 765. The toe off surface 775 may be configured as a continuous two-dimensional surface, such as a radius surface, a flat surface, and/or a combination of one or more radius surfaces and/or one or more flat surfaces. In an embodiment, the toe off surface 775 may be configured as a two-dimensional radius that is tangent to the ground contacting surface 760 and may extend towards the shoe facing surface 765.

The central portion 715 may include one or more central locating apertures 730 and a central width 755. The small flat directional gait modifier outsole 700 may include a plurality of slots 780 and a corresponding plurality of stress relief apertures 785.

The small flat directional gait modifier outsole 700 may be configured so that the hindfoot height 735 is generally equal to the forefoot height 745. The small flat directional gait modifier outsole 700 may further be configured so that the hindfoot width 740 and the forefoot width 750 are generally equal. The small flat directional gait modifier outsole 700 may further be configured so that the hindfoot width 740 and the forefoot width 750 are greater than the central width 755. The small flat directional gait modifier outsole 700 may further be configured so that heel touch surface 770 is generally congruent to the toe off surface 775.

In an embodiment, the small flat directional gait modifier outsole 700 may be one of a set of differently sized gait modifier outsoles 300 each having a different hindfoot height 735, a different forefoot height 745, a different hindfoot width 740, a different forefoot width 750, a different central width 755, a different heel touch surface 770, a different toe off surface 775, and/or a different outsole length 790.

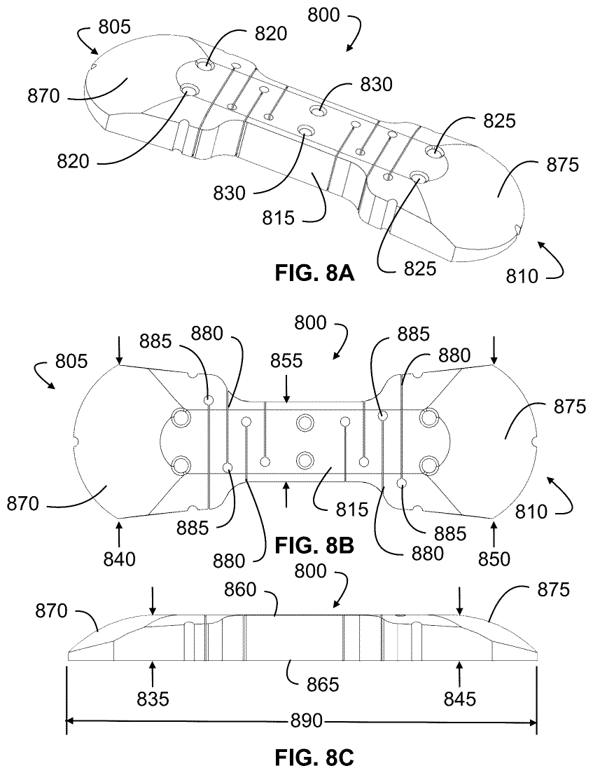

FIG. 8A is a perspective view of a small flat omni gait modifier outsole 800 according to an embodiment of the present disclosure. FIG. 8B is a bottom view of the small flat omni gait modifier outsole 800 and FIG. 8C is a side view of the small flat omni gait modifier outsole 800. The small flat omni gait modifier outsole 800 may include similar functions and features previously described for the gait modifier outsole 300. Conversely, the gait modifier outsole 300 may include similar functions and features as the small flat omni gait modifier outsole 800 described below.

The various features of the small flat omni gait modifier outsole 800 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the small flat omni gait modifier outsole 800 will be set forth below.

The small flat omni gait modifier outsole 800 may include a hindfoot portion 805, a forefoot portion 810, and a central portion 815 connecting the hindfoot portion 805 and the forefoot portion 810. The small flat omni gait modifier outsole 800 may further include a ground contacting surface 860, a shoe facing surface 865, and an outsole length 890. The outsole length 890 may be sized to be used in conjunction with a gait modifier shoe 100 sized between size 5 and size 9.5.

The hindfoot portion 805 may include one or more hindfoot locating apertures 820, a hindfoot height 835, a hindfoot width 840, and a heel touch surface 870. The heel touch surface 870 may be located on the hindfoot portion 805 and may extend, at least partially, between the ground contacting surface 860 and the shoe facing surface 865. The heel touch surface 870 may be configured as a continuous three-dimensional surface, such as a spherical radius surface, a cylindrical surface, and/or a combination of one or more spherical radius surfaces and/or one or more cylindrical surfaces. In an embodiment, the heel touch surface 870 may be configured as a three-dimensional spherical radius that is tangent to the ground contacting surface 860 and may extend towards the shoe facing surface 865. The three-dimensional surface may be configured as an omnidirectional surface.

The forefoot portion 810 may include one or more forefoot locating apertures 825, a forefoot height 845, a forefoot width 850, and a toe off surface 875. The toe off surface 875 may be configured as a continuous three-dimensional surface, such as a spherical radius surface, a cylindrical surface, and/or a combination of one or more spherical radius surfaces and/or one or more cylindrical surfaces. In an embodiment, the toe off surface 875 may be configured as a three-dimensional spherical radius that is tangent to the ground contacting surface 860 and may extend towards the shoe facing surface 865.

The central portion 815 may include one or more central locating apertures 830 and a central width 855. The small flat omni gait modifier outsole 800 may include a plurality of slots 880 and a corresponding plurality of stress relief apertures 885.

The small flat omni gait modifier outsole 800 may be configured so that the hindfoot height 835 is generally equal to the forefoot height 845. The small flat omni gait modifier outsole 800 may further be configured so that the hindfoot width 840 and the forefoot width 850 are generally equal. The small flat omni gait modifier outsole 800 may further be configured so that the hindfoot width 840 and the forefoot width 850 are greater than the central width 855. The small flat omni gait modifier outsole 800 may further be configured so that heel touch surface 870 is generally congruent to the toe off surface 875.

In an embodiment, the small flat omni gait modifier outsole 800 may be one of a set of differently sized gait modifier outsoles 300 each having a different hindfoot height 835, a different forefoot height 845, a different hindfoot width 840, a different forefoot width 850, a different central width 855, a different heel touch surface 870, a different toe off surface 875, and/or a different outsole length 890.

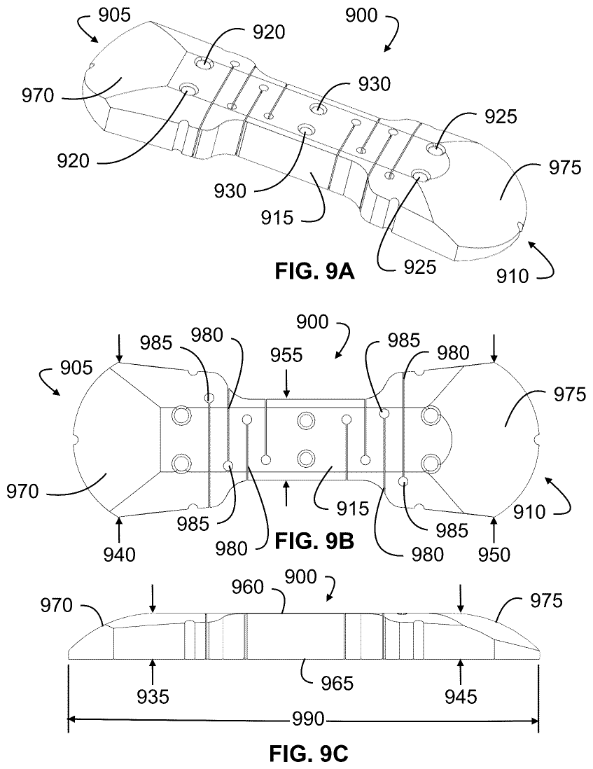

FIG. 9A is a perspective view of a small flat hybrid gait modifier outsole 900 according to an embodiment of the present disclosure. FIG. 9B is a bottom view of the small flat hybrid gait modifier outsole 900 and FIG. 9C is a side view of the small flat hybrid gait modifier outsole 900. The small flat hybrid gait modifier outsole 900 may include similar functions and features previously described for the gait modifier outsole 300. Conversely, the gait modifier outsole 300 may include similar functions and features as the small flat hybrid gait modifier outsole 900 described below.

The various features of the small flat hybrid gait modifier outsole 900 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the small flat hybrid gait modifier outsole 900 will be set forth below.

The small flat hybrid gait modifier outsole 900 may include a hindfoot portion 905, a forefoot portion 910, and a central portion 915 connecting the hindfoot portion 905 and the forefoot portion 910. The small flat hybrid gait modifier outsole 900 may further include a ground contacting surface 960, a shoe facing surface 965, and an outsole length 990. The outsole length 990 may be sized to be used in conjunction with a gait modifier shoe 100 sized between size 5 and size 9.5.

The hindfoot portion 905 may include one or more hindfoot locating apertures 920, a hindfoot height 935, a hindfoot width 940, and a heel touch surface 970. The heel touch surface 970 may be located on the hindfoot portion 905 and may extend, at least partially, between the ground contacting surface 960 and the shoe facing surface 965. The heel touch surface 970 may be configured as a continuous two-dimensional surface, such as a radius surface, a flat surface, and/or a combination of one or more radius surfaces and/or one or more flat surfaces. In an embodiment, the heel touch surface 970 may be configured as a two-dimensional radius that is tangent to the ground contacting surface 960 and may extend towards the shoe facing surface 965.

The forefoot portion 910 may include one or more forefoot locating apertures 925, a forefoot height 945, a forefoot width 950, and a toe off surface 975. The toe off surface 975 may be configured as a continuous three-dimensional surface, such as a spherical radius surface, a cylindrical surface, and/or a combination of one or more spherical radius surfaces and/or one or more cylindrical surfaces. In an embodiment, the toe off surface 975 may be configured as a three-dimensional spherical radius that is tangent to the ground contacting surface 960 and may extend towards the shoe facing surface 965. The two-dimensional surface may be configured as a unidirectional surface. The three-dimensional surface may be configured as an omnidirectional surface. The unidirectional surface may facilitate more effective control of the heel strike and/or toe off motion during a gait cycle than the omnidirectional surface.

The central portion 915 may include one or more central locating apertures 930 and a central width 955. The small flat hybrid gait modifier outsole 900 may include a plurality of slots 980 and a corresponding plurality of stress relief apertures 985.

The small flat hybrid gait modifier outsole 900 may be configured so that the hindfoot height 935 is generally equal to the forefoot height 945. The small flat hybrid gait modifier outsole 900 may further be configured so that the hindfoot width 940 and the forefoot width 950 are generally equal. The small flat hybrid gait modifier outsole 900 may further be configured so that the hindfoot width 940 and the forefoot width 950 are greater than the central width 955. The small flat hybrid gait modifier outsole 900 may further be configured so that heel touch surface 970 is configured as a two-dimension surface and the toe off surface 975 is configured as a three-dimensional surface.

In an embodiment, the small flat hybrid gait modifier outsole 900 may be one of a set of differently sized gait modifier outsoles 300 each having a different hindfoot height 935, a different forefoot height 945, a different hindfoot width 940, a different forefoot width 950, a different central width 955, a different heel touch surface 970, a different toe off surface 975, and/or a different outsole length 990.

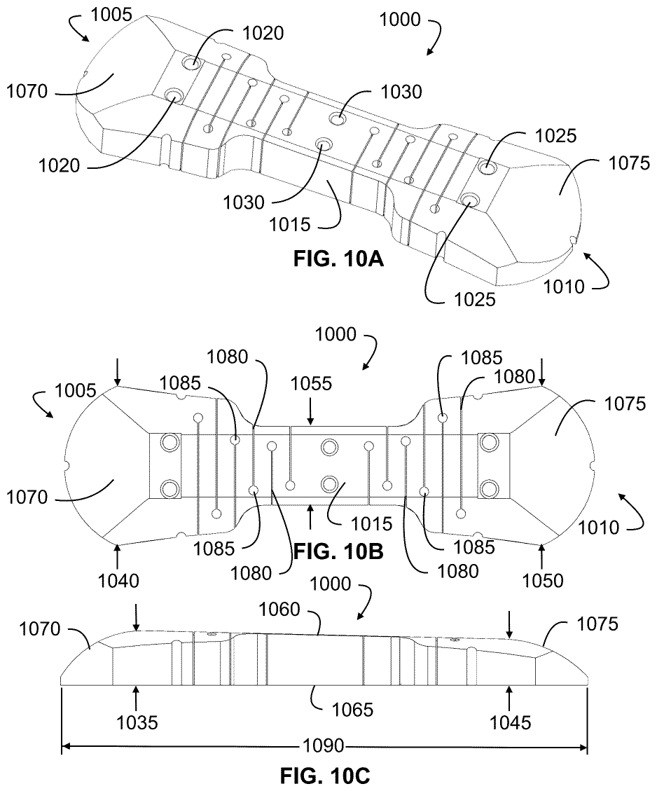

FIG. 10A is a perspective view of a large transition directional gait modifier outsole 1000 according to an embodiment of the present disclosure. FIG. 10B is a bottom view of the large transition directional gait modifier outsole 1000 and FIG. 10C is a side view of the large transition directional gait modifier outsole 1000. The large transition directional gait modifier outsole 1000 may include similar functions and features previously described for the gait modifier outsole 300. Conversely, the gait modifier outsole 300 may include similar functions and features as the large transition directional gait modifier outsole 1000 described below.

The various features of the large transition directional gait modifier outsole 1000 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the large transition directional gait modifier outsole 1000 will be set forth below.

The large transition directional gait modifier outsole 1000 may include a hindfoot portion 1005, a forefoot portion 1010, and a central portion 1015 connecting the hindfoot portion 1005 and the forefoot portion 1010. The large transition directional gait modifier outsole 1000 may further include a ground contacting surface 1060, a shoe facing surface 1065, and an outsole length 1090. The outsole length 1090 may be sized to be used in conjunction with a gait modifier shoe 100 sized between size 9 and size 15.

The hindfoot portion 1005 may include one or more hindfoot locating apertures 1020, a hindfoot height 1035, a hindfoot width 1040, and a heel touch surface 1070. The heel touch surface 1070 may be located on the hindfoot portion 1005 and may extend, at least partially, between the ground contacting surface 1060 and the shoe facing surface 1065. The heel touch surface 1070 may be configured as a continuous two-dimensional surface, such as a radius surface, a flat surface, and/or a combination of one or more radius surfaces and/or one or more flat surfaces. In an embodiment, the heel touch surface 1070 may be configured as a two-dimensional radius that is tangent to the ground contacting surface 1060 and may extend towards the shoe facing surface 1065. The two-dimensional surface may be configured as a unidirectional surface. The unidirectional surface may facilitate control of the heel strike and/or toe off motion during a gait cycle.

The forefoot portion 1010 may include one or more forefoot locating apertures 1025, a forefoot height 1045, a forefoot width 1050, and a toe off surface 1075. The toe off surface 1075 may be located on the forefoot portion 1010 and may extend, at least partially, between the ground contacting surface 1060 and the shoe facing surface 1065. The toe off surface 1075 may be configured as a continuous two-dimensional surface, such as a radius surface, a flat surface, and/or a combination of one or more radius surfaces and/or one or more flat surfaces. In an embodiment, the toe off surface 1075 may be configured as a two-dimensional radius that is tangent to the ground contacting surface 1060 and may extend towards the shoe facing surface 1065.

The central portion 1015 may include one or more central locating apertures 1030 and a central width 1055. The large transition directional gait modifier outsole 1000 may include a plurality of slots 1080 and a corresponding plurality of stress relief apertures 1085.

The large transition directional gait modifier outsole 1000 may be configured so that the hindfoot height 1035 is greater than the forefoot height 1045. The large transition directional gait modifier outsole 1000 may further be configured so that the hindfoot width 1040 and the forefoot width 1050 are generally equal. The large transition directional gait modifier outsole 1000 may further be configured so that the hindfoot width 1040 and the forefoot width 1050 are greater than the central width 1055. The large transition directional gait modifier outsole 1000 may further be configured so that heel touch surface 1070 is generally congruent to the toe off surface 1075.

In an embodiment, the large transition directional gait modifier outsole 1000 may be one of a set of differently sized gait modifier outsoles 300 each having a different hindfoot height 1035, a different forefoot height 1045, a different hindfoot width 1040, a different forefoot width 1050, a different central width 1055, a different heel touch surface 1070, a different toe off surface 1075, and/or a different outsole length 1090.

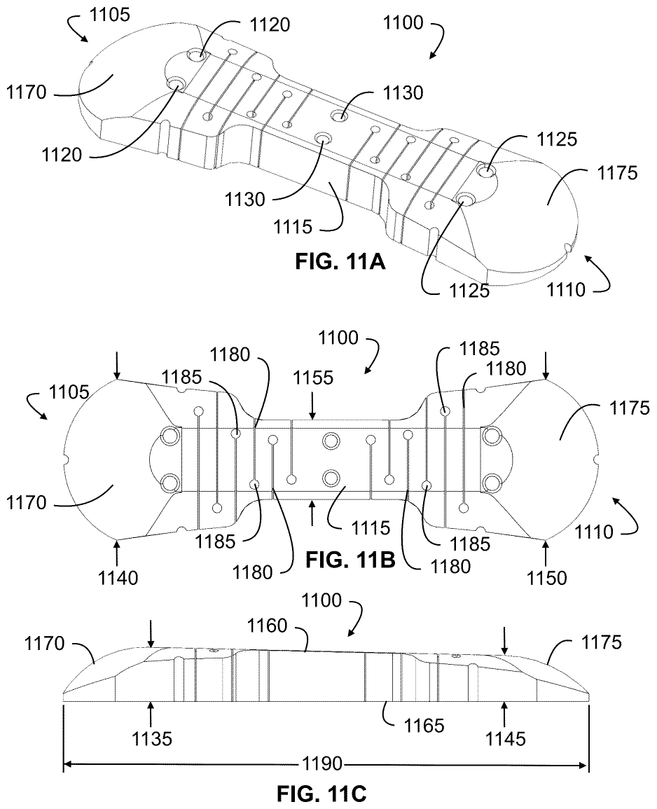

FIG. 11A is a perspective view of a large transition omni gait modifier outsole 1100 according to an embodiment of the present disclosure. FIG. 11B is a bottom view of the large transition omni gait modifier outsole 1100 and FIG. 11C is a side view of the large transition omni gait modifier outsole 1100. The large transition omni gait modifier outsole 1100 may include similar functions and features previously described for the gait modifier outsole 300. Conversely, the gait modifier outsole 300 may include similar functions and features as the large transition omni gait modifier outsole 1100 described below.

The various features of the large transition omni gait modifier outsole 1100 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the large transition omni gait modifier outsole 1100 will be set forth below.

The large transition omni gait modifier outsole 1100 may include a hindfoot portion 1105, a forefoot portion 1110, and a central portion 1115 connecting the hindfoot portion 1105 and the forefoot portion 1110. The large transition omni gait modifier outsole 1100 may further include a ground contacting surface 1160, a shoe facing surface 1165, and an outsole length 1190. The outsole length 1190 may be sized to be used in conjunction with a gait modifier shoe 100 sized between size 9 and size 15.

The hindfoot portion 1105 may include one or more hindfoot locating apertures 1120, a hindfoot height 1135, a hindfoot width 1140, and a heel touch surface 1170. The heel touch surface 1170 may be located on the hindfoot portion 1105 and may extend, at least partially, between the ground contacting surface 1160 and the shoe facing surface 1165. The heel touch surface 1170 may be configured as a continuous three-dimensional surface, such as a spherical radius surface, a cylindrical surface, and/or a combination of one or more spherical radius surfaces and/or one or more cylindrical surfaces. In an embodiment, the heel touch surface 1170 may be configured as a three-dimensional spherical radius that is tangent to the ground contacting surface 1160 and may extend towards the shoe facing surface 1165. The three-dimensional surface may be configured as an omnidirectional surface.

The forefoot portion 1110 may include one or more forefoot locating apertures 1125, a forefoot height 1145, a forefoot width 1150, and a toe off surface 1175. The toe off surface 1175 may be configured as a continuous three-dimensional surface, such as a spherical radius surface, a cylindrical surface, and/or a combination of one or more spherical radius surfaces and/or one or more cylindrical surfaces. In an embodiment, the toe off surface 1175 may be configured as a three-dimensional spherical radius that is tangent to the ground contacting surface 1160 and may extend towards the shoe facing surface 1165.

The central portion 1115 may include one or more central locating apertures 1130 and a central width 1155. The large transition omni gait modifier outsole 1100 may include a plurality of slots 1180 and a corresponding plurality of stress relief apertures 1185.

The large transition omni gait modifier outsole 1100 may be configured so that the hindfoot height 1135 is greater than the forefoot height 1145. The large transition omni gait modifier outsole 1100 may further be configured so that the hindfoot width 1140 and the forefoot width 1150 are generally equal. The large transition omni gait modifier outsole 1100 may further be configured so that the hindfoot width 1140 and the forefoot width 1150 are greater than the central width 1155. The large transition omni gait modifier outsole 1100 may further be configured so that heel touch surface 1170 is generally congruent to the toe off surface 1175.

In an embodiment, the large transition omni gait modifier outsole 1100 may be one of a set of differently sized gait modifier outsoles 300 each having a different hindfoot height 1135, a different forefoot height 1145, a different hindfoot width 1140, a different forefoot width 1150, a different central width 1155, a different heel touch surface 1170, a different toe off surface 1175, and/or a different outsole length 1190.

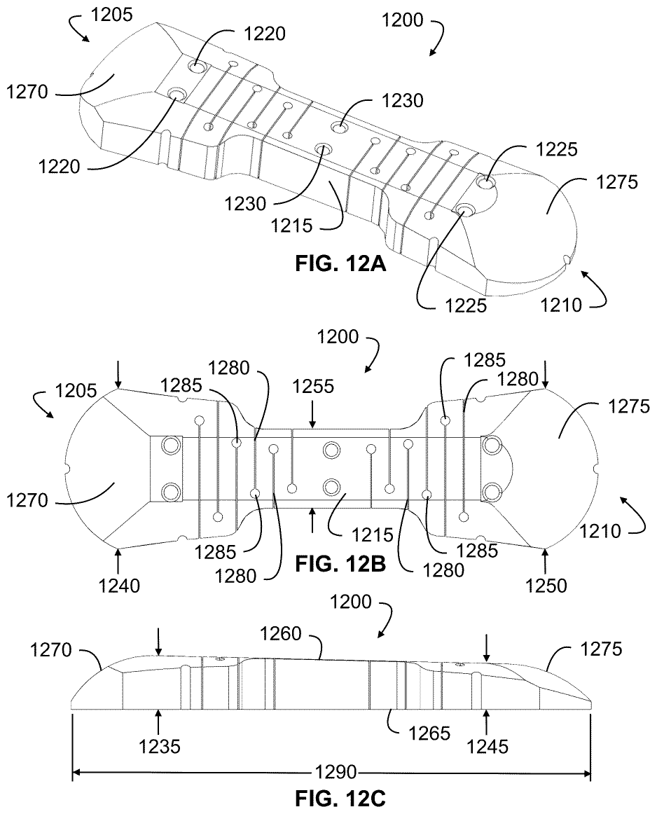

FIG. 12A is a perspective view of a large transition hybrid gait modifier outsole 1200 according to an embodiment of the present disclosure. FIG. 12B is a bottom view of the large transition hybrid gait modifier outsole 1200 and FIG. 12C is a side view of the large transition hybrid gait modifier outsole 1200. The large transition hybrid gait modifier outsole 1200 may include similar functions and features previously described for the gait modifier outsole 300. Conversely, the gait modifier outsole 300 may include similar functions and features as the large transition hybrid gait modifier outsole 1200 described below.

The various features of the large transition hybrid gait modifier outsole 1200 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the large transition hybrid gait modifier outsole 1200 will be set forth below.

The large transition hybrid gait modifier outsole 1200 may include a hindfoot portion 1205, a forefoot portion 1210, and a central portion 1215 connecting the hindfoot portion 1205 and the forefoot portion 1210. The large transition hybrid gait modifier outsole 1200 may further include a ground contacting surface 1260, a shoe facing surface 1265, and an outsole length 1290. The outsole length 1290 may be sized to be used in conjunction with a gait modifier shoe 100 sized between size 9 and size 15.

The hindfoot portion 1205 may include one or more hindfoot locating apertures 1220, a hindfoot height 1235, a hindfoot width 1240, and a heel touch surface 1270. The heel touch surface 1270 may be located on the hindfoot portion 1205 and may extend, at least partially, between the ground contacting surface 1260 and the shoe facing surface 1265. The heel touch surface 1270 may be configured as a continuous two-dimensional surface, such as a radius surface, a flat surface, and/or a combination of one or more radius surfaces and/or one or more flat surfaces. In an embodiment, the heel touch surface 1270 may be configured as a two-dimensional radius that is tangent to the ground contacting surface 1260 and may extend towards the shoe facing surface 1265.

The forefoot portion 1210 may include one or more forefoot locating apertures 1225, a forefoot height 1245, a forefoot width 1250, and a toe off surface 1275. The toe off surface 1275 may be configured as a continuous three-dimensional surface, such as a spherical radius surface, a cylindrical surface, and/or a combination of one or more spherical radius surfaces and/or one or more cylindrical surfaces. In an embodiment, the toe off surface 1275 may be configured as a three-dimensional spherical radius that is tangent to the ground contacting surface 1260 and may extend towards the shoe facing surface 1265. The two-dimensional surface may be configured as a unidirectional surface. The three-dimensional surface may be configured as an omnidirectional surface. The unidirectional surface may facilitate more effective control of the heel strike and/or toe off motion during a gait cycle than the omnidirectional surface.

The central portion 1215 may include one or more central locating apertures 1230 and a central width 1255. The large transition hybrid gait modifier outsole 1200 may include a plurality of slots 1280 and a corresponding plurality of stress relief apertures 1285.

The large transition hybrid gait modifier outsole 1200 may be configured so that the hindfoot height 1235 is greater than the forefoot height 1245. The large transition hybrid gait modifier outsole 1200 may further be configured so that the hindfoot width 1240 and the forefoot width 1250 are generally equal. The large transition hybrid gait modifier outsole 1200 may further be configured so that the hindfoot width 1240 and the forefoot width 1250 are greater than the central width 1255. The large transition hybrid gait modifier outsole 1200 may further be configured so that heel touch surface 1270 is configured as a two-dimension surface and the toe off surface 1275 is configured as a three-dimensional surface.

In an embodiment, the large transition hybrid gait modifier outsole 1200 may be one of a set of differently sized gait modifier outsoles 300 each having a different hindfoot height 1235, a different forefoot height 1245, a different hindfoot width 1240, a different forefoot width 1250, a different central width 1255, a different heel touch surface 1270, a different toe off surface 1275, and/or a different outsole length 1290.

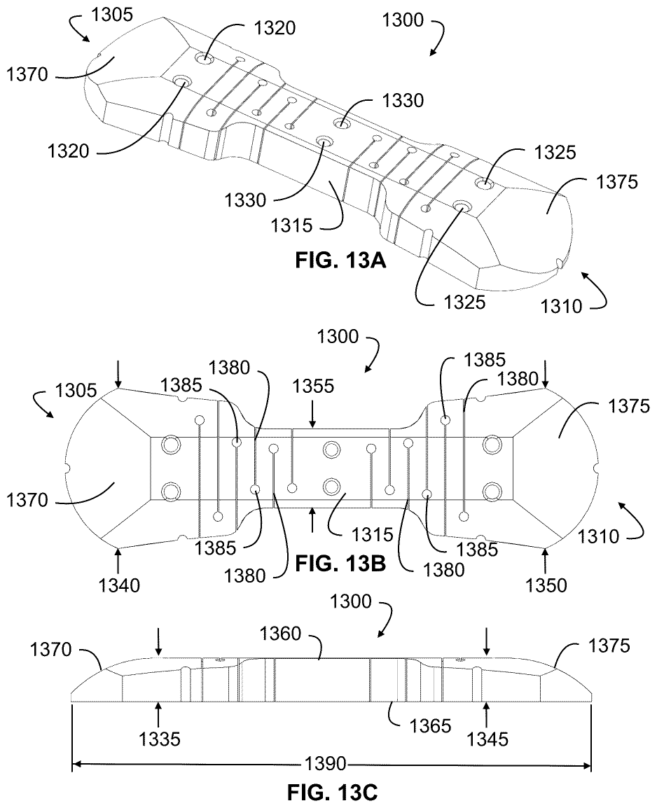

FIG. 13A is a perspective view of a large flat directional gait modifier outsole 1300 according to an embodiment of the present disclosure. FIG. 13B is a bottom view of the large flat directional gait modifier outsole 1300 and FIG. 13C is a side view of the large flat directional gait modifier outsole 1300. The large flat directional gait modifier outsole 1300 may include similar functions and features previously described for the gait modifier outsole 300. Conversely, the gait modifier outsole 300 may include similar functions and features as the large flat directional gait modifier outsole 1300 described below.

The various features of the large flat directional gait modifier outsole 1300 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the large flat directional gait modifier outsole 1300 will be set forth below.

The large flat directional gait modifier outsole 1300 may include a hindfoot portion 1305, a forefoot portion 1310, and a central portion 1315 connecting the hindfoot portion 1305 and the forefoot portion 1310. The large flat directional gait modifier outsole 1300 may further include a ground contacting surface 1360, a shoe facing surface 1365, and an outsole length 1390. The outsole length 1390 may be sized to be used in conjunction with a gait modifier shoe 100 sized between size 9 and size 15.

The hindfoot portion 1305 may include one or more hindfoot locating apertures 1320, a hindfoot height 1335, a hindfoot width 1340, and a heel touch surface 1370. The heel touch surface 1370 may be located on the hindfoot portion 1305 and may extend, at least partially, between the ground contacting surface 1360 and the shoe facing surface 1365. The heel touch surface 1370 may be configured as a continuous two-dimensional surface, such as a radius surface, a flat surface, and/or a combination of one or more radius surfaces and/or one or more flat surfaces. In an embodiment, the heel touch surface 1370 may be configured as a two-dimensional radius that is tangent to the ground contacting surface 1360 and may extend towards the shoe facing surface 1365. The two-dimensional surface may be configured as a unidirectional surface. The unidirectional surface may facilitate control of the heel strike and/or toe off motion during a gait cycle.

The forefoot portion 1310 may include one or more forefoot locating apertures 1325, a forefoot height 1345, a forefoot width 1350, and a toe off surface 1375. The toe off surface 1375 may be located on the forefoot portion 1310 and may extend, at least partially, between the ground contacting surface 1360 and the shoe facing surface 1365. The toe off surface 1375 may be configured as a continuous two-dimensional surface, such as a radius surface, a flat surface, and/or a combination of one or more radius surfaces and/or one or more flat surfaces. In an embodiment, the toe off surface 1375 may be configured as a two-dimensional radius that is tangent to the ground contacting surface 1360 and may extend towards the shoe facing surface 1365.

The central portion 1315 may include one or more central locating apertures 1330 and a central width 1355. The large flat directional gait modifier outsole 1300 may include a plurality of slots 1380 and a corresponding plurality of stress relief apertures 1385.

The large flat directional gait modifier outsole 1300 may be configured so that the hindfoot height 1335 is generally equal to the forefoot height 1345. The large flat directional gait modifier outsole 1300 may further be configured so that the hindfoot width 1340 and the forefoot width 1350 are generally equal. The large flat directional gait modifier outsole 1300 may further be configured so that the hindfoot width 1340 and the forefoot width 1350 are greater than the central width 1355. The large flat directional gait modifier outsole 1300 may further be configured so that heel touch surface 1370 is generally congruent to the toe off surface 1375.

In an embodiment, the large flat directional gait modifier outsole 1300 may be one of a set of differently sized gait modifier outsoles 300 each having a different hindfoot height 1335, a different forefoot height 1345, a different hindfoot width 1340, a different forefoot width 1350, a different central width 1355, a different heel touch surface 1370, a different toe off surface 1375, and/or a different outsole length 1390.

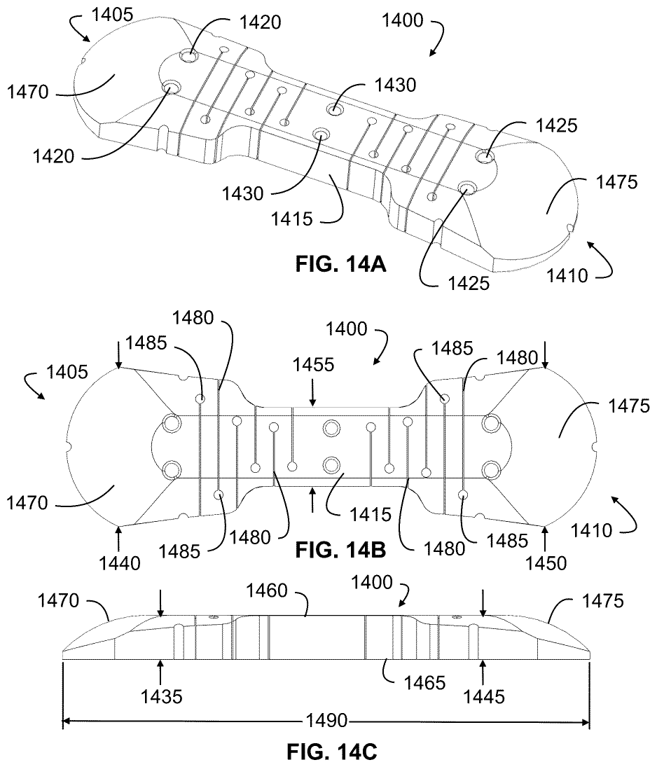

FIG. 14A is a perspective view of a large flat omni gait modifier outsole 1400 according to an embodiment of the present disclosure. FIG. 14B is a bottom view of the large flat omni gait modifier outsole 1400 and FIG. 14C is a side view of the large flat omni gait modifier outsole 1400. The large flat omni gait modifier outsole 1400 may include similar functions and features previously described for the gait modifier outsole 300. Conversely, the gait modifier outsole 300 may include similar functions and features as the large flat omni gait modifier outsole 1400 described below.

The various features of the large flat omni gait modifier outsole 1400 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the large flat omni gait modifier outsole 1400 will be set forth below.

The large flat omni gait modifier outsole 1400 may include a hindfoot portion 1405, a forefoot portion 1410, and a central portion 1415 connecting the hindfoot portion 1405 and the forefoot portion 1410. The large flat omni gait modifier outsole 1400 may further include a ground contacting surface 1460, a shoe facing surface 1465, and an outsole length 1490. The outsole length 1490 may be sized to be used in conjunction with a gait modifier shoe 100 sized between size 9 and size 15.

The hindfoot portion 1405 may include one or more hindfoot locating apertures 1420, a hindfoot height 1435, a hindfoot width 1440, and a heel touch surface 1470. The heel touch surface 1470 may be located on the hindfoot portion 1405 and may extend, at least partially, between the ground contacting surface 1460 and the shoe facing surface 1465. The heel touch surface 1470 may be configured as a continuous three-dimensional surface, such as a spherical radius surface, a cylindrical surface, and/or a combination of one or more spherical radius surfaces and/or one or more cylindrical surfaces. In an embodiment, the heel touch surface 1470 may be configured as a three-dimensional spherical radius that is tangent to the ground contacting surface 1460 and may extend towards the shoe facing surface 1465. The three-dimensional surface may be configured as an omnidirectional surface.

The forefoot portion 1410 may include one or more forefoot locating apertures 1425, a forefoot height 1445, a forefoot width 1450, and a toe off surface 1475. The toe off surface 1475 may be configured as a continuous three-dimensional surface, such as a spherical radius surface, a cylindrical surface, and/or a combination of one or more spherical radius surfaces and/or one or more cylindrical surfaces. In an embodiment, the toe off surface 1475 may be configured as a three-dimensional spherical radius that is tangent to the ground contacting surface 1460 and may extend towards the shoe facing surface 1465.

The central portion 1415 may include one or more central locating apertures 1430 and a central width 1455. The large flat omni gait modifier outsole 1400 may include a plurality of slots 1480 and a corresponding plurality of stress relief apertures 1485.

The large flat omni gait modifier outsole 1400 may be configured so that the hindfoot height 1435 is generally equal to the forefoot height 1445. The large flat omni gait modifier outsole 1400 may further be configured so that the hindfoot width 1440 and the forefoot width 1450 are generally equal. The large flat omni gait modifier outsole 1400 may further be configured so that the hindfoot width 1440 and the forefoot width 1450 are greater than the central width 1455. The large flat omni gait modifier outsole 1400 may further be configured so that heel touch surface 1470 is generally congruent to the toe off surface 1475.

In an embodiment, the large flat omni gait modifier outsole 1400 may be one of a set of differently sized gait modifier outsoles 300 each having a different hindfoot height 1435, a different forefoot height 1445, a different hindfoot width 1440, a different forefoot width 1450, a different central width 1455, a different heel touch surface 1470, a different toe off surface 1475, and/or a different outsole length 1490.

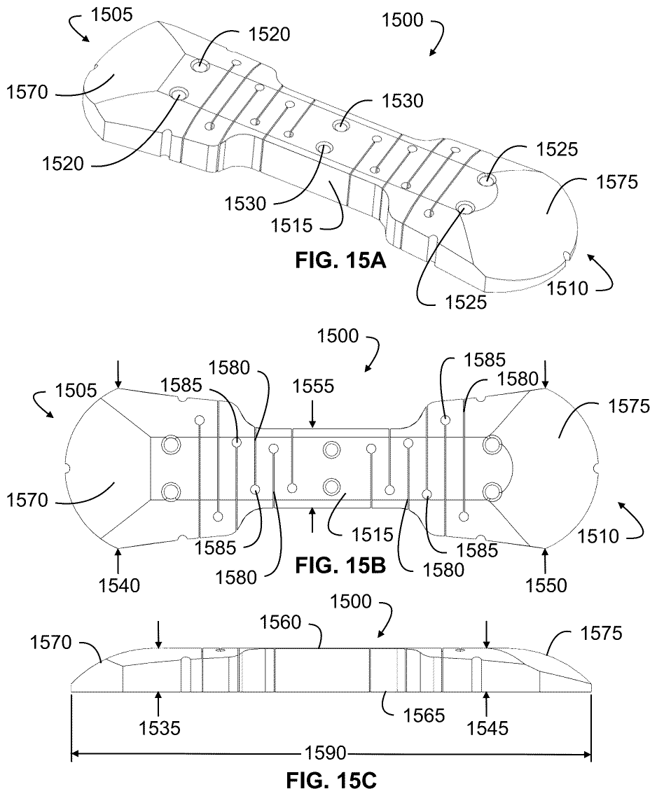

FIG. 15A is a perspective view of a large flat hybrid gait modifier outsole 1500 according to an embodiment of the present disclosure. FIG. 15B is a bottom view of the large flat hybrid gait modifier outsole 1500 and FIG. 15C is a side view of the large flat hybrid gait modifier outsole 1500. The large flat hybrid gait modifier outsole 1500 may include similar functions and features previously described for the gait modifier outsole 300. Conversely, the gait modifier outsole 300 may include similar functions and features as the large flat hybrid gait modifier outsole 1500 described below.

The various features of the large flat hybrid gait modifier outsole 1500 with similar reference numbers as those of the gait modifier outsole 300 and/or the small transition directional gait modifier outsole 400 may be similar in function and/or configuration to their counterparts of the gait modifier outsole 300 and/or the small transition directional gait modifier outsole 400. Differences between the gait modifier outsole 300 and/or the small transition directional gait modifier outsole 400 and the large flat hybrid gait modifier outsole 1500 will be set forth below.

The large flat hybrid gait modifier outsole 1500 may include a hindfoot portion 1505, a forefoot portion 1510, and a central portion 1515 connecting the hindfoot portion 1505 and the forefoot portion 1510. The large flat hybrid gait modifier outsole 1500 may further include a ground contacting surface 1560, a shoe facing surface 1565, and an outsole length 1590. The outsole length 1590 may be sized to be used in conjunction with a gait modifier shoe 100 sized between size 9 and size 15.

The hindfoot portion 1505 may include one or more hindfoot locating apertures 1520, a hindfoot height 1535, a hindfoot width 1540, and a heel touch surface 1570. The heel touch surface 1570 may be located on the hindfoot portion 1505 and may extend, at least partially, between the ground contacting surface 1560 and the shoe facing surface 1565. The heel touch surface 1570 may be configured as a continuous two-dimensional surface, such as a radius surface, a flat surface, and/or a combination of one or more radius surfaces and/or one or more flat surfaces. In an embodiment, the heel touch surface 1570 may be configured as a two-dimensional radius that is tangent to the ground contacting surface 1560 and may extend towards the shoe facing surface 1565.

The forefoot portion 1510 may include one or more forefoot locating apertures 1525, a forefoot height 1545, a forefoot width 1550, and a toe off surface 1575. The toe off surface 1575 may be configured as a continuous three-dimensional surface, such as a spherical radius surface, a cylindrical surface, and/or a combination of one or more spherical radius surfaces and/or one or more cylindrical surfaces. In an embodiment, the toe off surface 1575 may be configured as a three-dimensional spherical radius that is tangent to the ground contacting surface 1560 and may extend towards the shoe facing surface 1565. The two-dimensional surface may be configured as a unidirectional surface. The three-dimensional surface may be configured as an omnidirectional surface. The unidirectional surface may facilitate more effective control of the heel strike and/or toe off motion during a gait cycle than the omnidirectional surface. The omnidirectional surface may be configured to allow rotation of the gait modifier outsole 300 about a longitudinal axis of the gait modifier outsole 300 during ambulation.

The central portion 1515 may include one or more central locating apertures 1530 and a central width 1555. The large flat hybrid gait modifier outsole 1500 may include a plurality of slots 1580 and a corresponding plurality of stress relief apertures 1585.

The large flat hybrid gait modifier outsole 1500 may be configured so that the hindfoot height 1535 is generally equal to the forefoot height 1545. The large flat hybrid gait modifier outsole 1500 may further be configured so that the hindfoot width 1540 and the forefoot width 1550 are generally equal. The large flat hybrid gait modifier outsole 1500 may further be configured so that the hindfoot width 1540 and the forefoot width 1550 are greater than the central width 1555. The large flat hybrid gait modifier outsole 1500 may further be configured so that heel touch surface 1570 is configured as a two-dimension surface and the toe off surface 1575 is configured as a three-dimensional surface.

In an embodiment, the large flat hybrid gait modifier outsole 1500 may be one of a set of differently sized gait modifier outsoles 300 each having a different hindfoot height 1535, a different forefoot height 1545, a different hindfoot width 1540, a different forefoot width 1550, a different central width 1555, a different heel touch surface 1570, a different toe off surface 1575, and/or a different outsole length 1590.

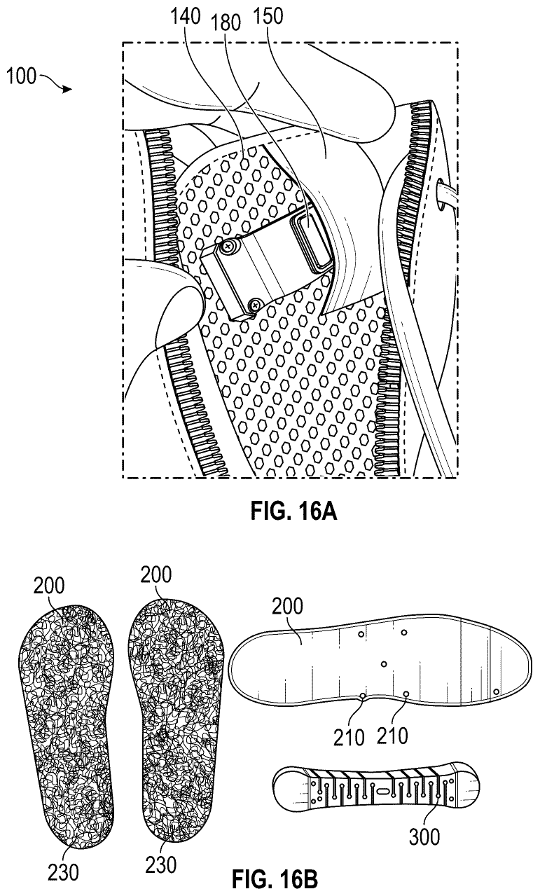

FIG. 16A is a perspective view of a gait modifier shoe 100 according to an embodiment of the present disclosure. The gait modifier shoe 100 may further include a pocket 150 located on the tongue 140. The pocket may be configured to receive a sensor 180. The pocket 150 and the sensor 180 may be configured so that the sensor remains within the pocket during ambulation of the patient while wearing the gait modifier shoe 100.

The sensor 180 may be configured as a motion sensor. The sensor 180 may include a storage media configured to record data from the sensor 180 including, but not limited to: step count, stride length, and/or other gait data. Additionally, or alternatively, the sensor 180 may include a wired connection to a storage media device configured to record data from the sensor 180 including, but not limited to: step count, stride length, and/or other gait data.

Additionally, or alternatively, the sensor 180 may be configured for a wireless connection to a storage media device configured to record data from the sensor 180 including, but not limited to: step count, stride length, and/or other gait data. The data from the sensor 180 may be included in a gait analysis to evaluate placement of the gait modifier outsole 300 on the gait modifier shoe 100. A position of the gait modifier outsole 300 on the gait modifier shoe 100 may be re-adjusted based on data from the sensor 180.

FIG. 16B includes top and bottom views of a plate 200 and a bottom view of a gait modifier outsole 300 according to an embodiment of the present disclosure. The plate 200 may further be configured to function as a stiffening plate in order to provide additional support to the patient and to help ensure the patient's gait is effectively directed through the gait modifier outsole 300. The plate 200 may be manufactured from metal and/or rigid plastic. The plate 200 may include a shoe facing side 220, a ground facing side 230, and one or more locating apertures 210.

The shoe facing side 220 may be configured to attach to the fastening surface 190 of the shoe outsole 160. The shoe facing side 220 may include a hook-and-loop fastener type surface, (ex: Velcro®). Additionally, or alternatively, the shoe facing side 220 may include other means for removably attaching the plate 200 to the shoe outsole 160, such as: magnets, removeable adhesive, clevis pins, cotter pins, linchpins, retaining rings, threaded fasteners, snaps, turn-button fasteners, clips, and/or another removably and securably attachable fastening mechanism. The shoe facing side 220 may include a fastening feature that is complementary to the fastening surface 190 so that the plate 200 may be removably and securably attachable to the shoe outsole 160.

The ground facing side 230 may be configured to attach to the shoe facing surface 365 of the gait modifier outsole 300. The ground facing side 230 may include a hook-and-loop fastener type surface, (ex: Velcro®). Additionally, or alternatively, the ground facing side 230 may include other means for removably attaching the gait modifier outsole 300 to the plate 200, such as: magnets, removeable adhesive, clevis pins, cotter pins, linchpins, retaining rings, threaded fasteners, snaps, turn-button fasteners, clips, and/or another removably and securably attachable fastening mechanism. The ground facing side 230 may include a fastening feature that is complementary to the shoe facing surface 365 so that the gait modifier outsole 300 may be removably and securably attachable to the plate 200.

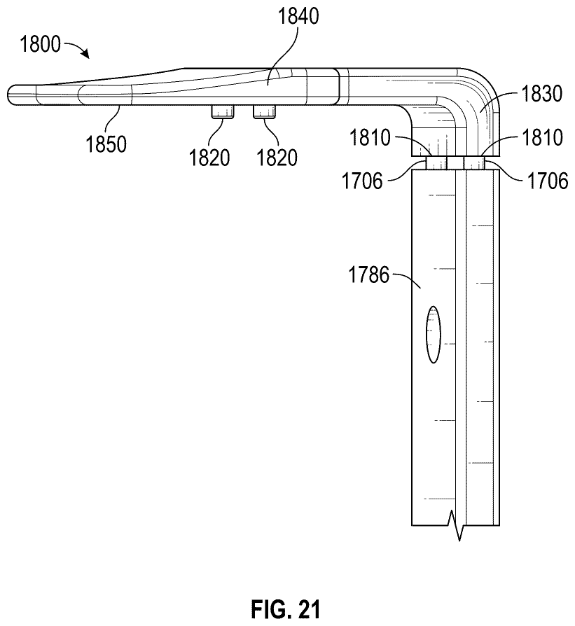

The one or more locating apertures 210 may be configured to receive the one or more plate locating features 1820 (as shown in FIG. 21). The one or more locating apertures 210 may facilitate alignment and/or positioning of the plate 200 relative to the gait modifier outsole 300. The one or more locating apertures 210 may further be configured to receive the one or more locating features 170 (as shown in FIG. 1). The one or more locating apertures 210 may further facilitate alignment and/or positioning of the plate 200 relative to the shoe outsole 160, thereby facilitating alignment and/or positioning of the gait modifier outsole 300 relative to the shoe outsole 160.

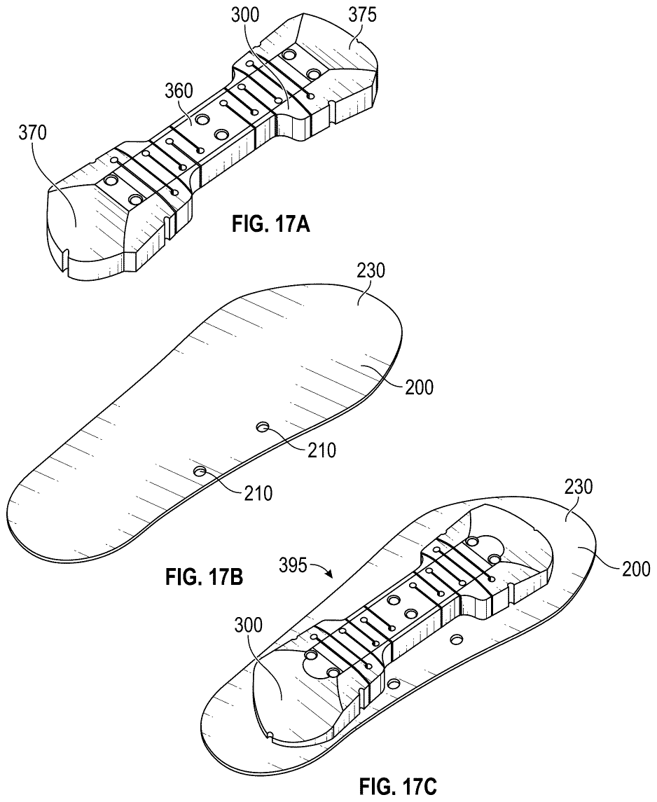

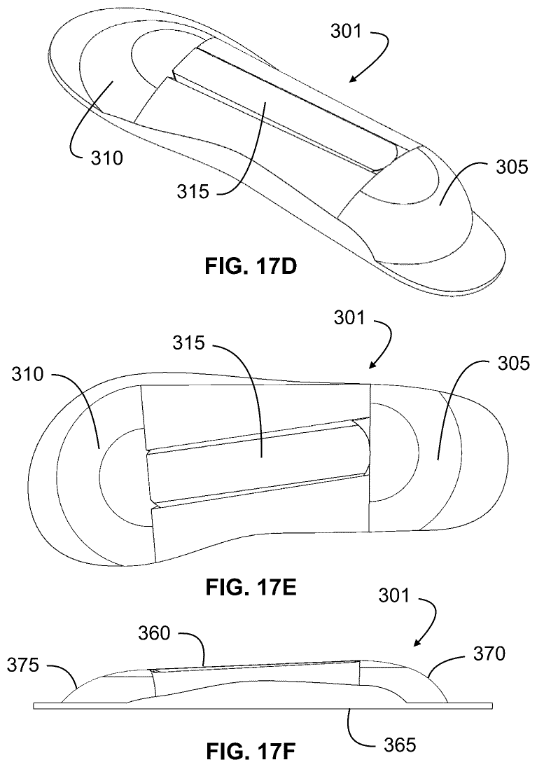

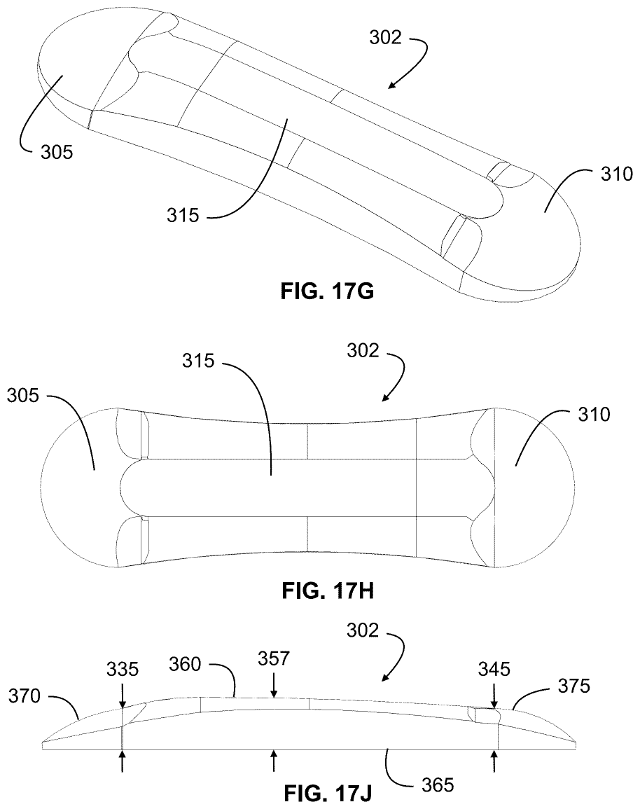

FIG. 17A is a perspective view of a gait modifier outsole 300 according to an embodiment of the present disclosure. FIG. 17B is a perspective view of a plate 200 according to an embodiment of the present disclosure. FIG. 17C is a perspective view of a gait modifier 395 according to an embodiment of the present disclosure. The gait modifier outsole 300 may further include a heel touch surface 370 and a toe off surface 375. The heel touch surface 370 may be configured so that, during a gait cycle, the heel touch surface 370 may be the first ground contacting surface of the gait modifier outsole 300. Additionally, the toe off surface 375 may be configured so that, during a gait cycle, the toe off surface 375 is the last ground contacting surface of the gait modifier outsole 300.

The gait modifier outsole 300 may include a neutral state and an adjusted state. In the neutral state, the hindfoot portion 305, the forefoot portion 310, and the central portion 315 may be generally aligned along a central longitudinal axis. Additionally, or alternatively, in the neutral state, the plurality of slots 380 may be generally unexpanded and/or generally uncontracted. In the adjusted state the gait modifier outsole 300 may be expanded, contracted, rotated, and/or angled relative to the neutral state.

With the gait modifier outsole 300 in the adjusted state, the gait modifier outsole 300 may be securably attached to the plate 200 thereby securing the gait modifier outsole 300 in the adjusted state and inhibiting any subsequent adjustment. The gait modifier 395 may include the gait modifier outsole 300 and the plate 200, wherein the gait modifier outsole 300 is in the adjusted state and the gait modifier outsole 300 is secured to the plate 200. The gait modifier outsole 300 may be removed from the plate 200, re-adjusted to alter the adjustment state, and then re-attached to the plate 200. The gait modifier 395 may be removably and securably attached to the gait modifier shoe 100. The gait modifier 395 may be removable from the gait modifier shoe 100 without altering the adjusted state of the gait modifier outsole 300. The gait modifier 395 may be re-attachable to the gait modifier shoe 100 without altering the adjusted state of the gait modifier outsole 300.

FIG. 17D is a perspective view of a patient specific outsole 301 according to an embodiment of the present disclosure. FIG. 17E is a bottom view of the patient specific outsole 301 and FIG. 17F is a side view of the patient specific outsole 301. Adjustment specifications of an adjusted gait modifier outsole 300 may be used to manufacture a patient specific outsole 301. Additionally, or alternatively, a three-dimensional scan may be performed on the gait modifier outsole 300 to create a three-dimensional model that may be used to manufacture a patient specific outsole 301. The patient specific outsole 301 may be manufactured using additive manufacturing, subtractive manufacturing, molding, and/or other manufacturing techniques known in the art.

The patient specific outsole 301 may be manufactured to include varying material properties within the gait modifier outsole 300. Through additive manufacturing, the porosity, modulus, and/or material density may vary. The porosity, modulus, and/or material density may be varied along an anterior-posterior axis, along a medial-lateral axis, and/or along a cephalad-caudal axis.

The patient specific outsole 301 may include a patient's name and/or other patient specific information embossed and/or debossed on a surface of the patient specific outsole 301. The patient specific outsole 301 may further include specific layers that indicate to the patient when the patient specific outsole 301 has worn and needs to be replaced. The patient specific outsole 301 may be configured to attach to the shoe outsole 160. Additionally, or alternatively, the patient specific outsole 301 may be configured to securably attach to the plate 200 and the plate 200 may then securably attach to the shoe outsole 160.

The patient specific outsole 301 may include a hindfoot portion 305, a forefoot portion 310, and a central portion 315. The patient specific outsole 301 may not include slots 380 to facilitate further adjustment. The patient specific outsole 301 may be configured as a single monolithic outsole including a hindfoot portion and a forefoot portion. The patient specific outsole 301 may further include a ground contacting surface 360, a shoe facing surface 365, a heel touch surface 370, and a toe off surface 375.

The heel touch surface 370 may be configured so that, during a gait cycle, the heel touch surface 370 may be the first ground contacting surface of the patient specific outsole 301. Additionally, the toe off surface 375 may be configured so that, during a gait cycle, the toe off surface 375 is the last ground contacting surface of the patient specific outsole 301.

FIG. 17G is a perspective view of a rocker outsole 302 according to an embodiment of the present disclosure. FIG. 17H is a bottom view of the rocker outsole 302 and FIG. 17J is a side view of the rocker outsole 302. The rocker outsole 302 may include similar features to the patient specific outsole 301.

The rocker outsole 302 may include a hindfoot portion 305, a forefoot portion 310, and a central portion 315 connecting the hindfoot portion 305 and the forefoot portion 310. The central portion 315 of the rocker outsole 302 may be configured with a curved profile. The central portion 315 may include a curved shape allowing for a smoother transition from heel strike to toe off. The curved profile of the rocker outsole 302 may reduce excessive ankle movement and may help propel the foot forward more efficiently during ambulation.

The rocker outsole 302 may further include a ground contacting surface 360, a shoe facing surface 365, a hindfoot height 335, a forefoot height 345, and a central height 357. The hindfoot height 335 may be the maximum distance between the ground contacting surface 360 and the shoe facing surface 365 in the hindfoot portion 305. The forefoot height 345 may be the maximum distance between the ground contacting surface 360 and the shoe facing surface 365 in the forefoot portion 310.

The central height 357 may be the maximum distance between the ground contacting surface 360 and the shoe facing surface 365 in the central portion 315. The central height 357 may be greater than the hindfoot height 335 and the forefoot height 345. The central portion 315 may be generally curved and may include a single radius or multiple radii. The central portion 315 may be generally tangent to the heel touch surface 370 and/or the toe off surface 375.

The central portion 315 may include a single radius, a spline, and/or a polyline. A center point of the central portion 315 may be centered relative to a sagittal plane and a coronal plane. Alternatively, the center point may be located anteriorly or posteriorly relative to the coronal plane. Additionally, or alternatively, the center point may be located medially or laterally relative to the sagittal plane.

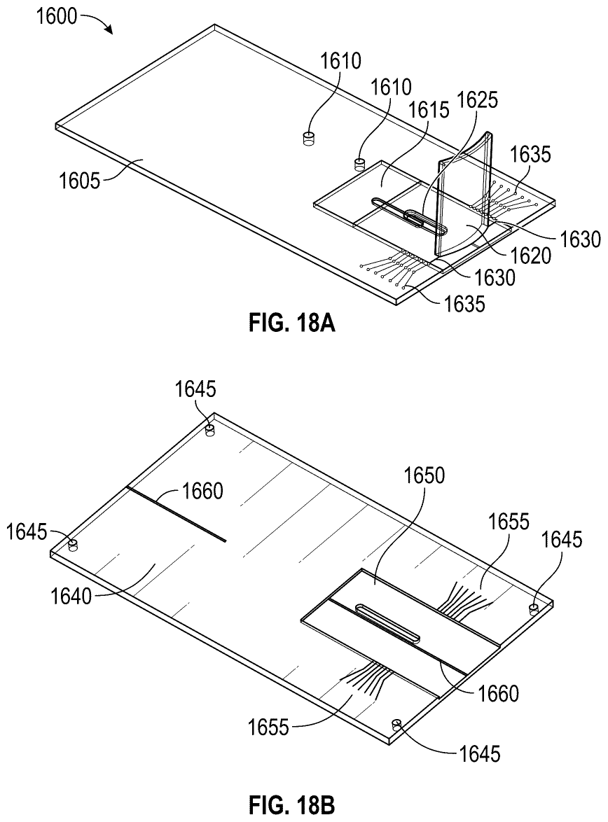

FIG. 18A is a perspective view of a baseline fixture 1600 according to an embodiment of the present disclosure. The baseline fixture 1600 may be configured to receive a patient's foot to facilitate identifying and/or recording one or more locations of one or more anatomic features of the patient. The baseline fixture 1600 may be used to identify and/or record locations of a patient's metatarsophalangeal joint (MTP) and/or medial malleolus. The recorded locations of the baseline fixture 1600 may be used to adjust a gait modifier outsole 300 to a baseline state. In an embodiment, the baseline state of the gait modifier outsole 300 may be different than the adjusted state of the gait modifier outsole 300.

The baseline fixture 1600 may be manufactured from transparent and/or translucent polymer, for example: plexiglass, acrylic, butyrate, polycarbonate, PVC, PET, and/or PETG. Alternatively, the baseline fixture 1600 may be manufactured of other transparent and/or translucent material that enables the gait modifier outsole 300 to be visible with the baseline fixture 1600 aligned above the gait modifier outsole 300. Additionally, or alternatively, the baseline fixture 1600 may be manufactured with cut outs, windows, perforations, and/or lattice patterns that facilitate visualization of the gait modifier outsole 300 through the baseline fixture 1600.

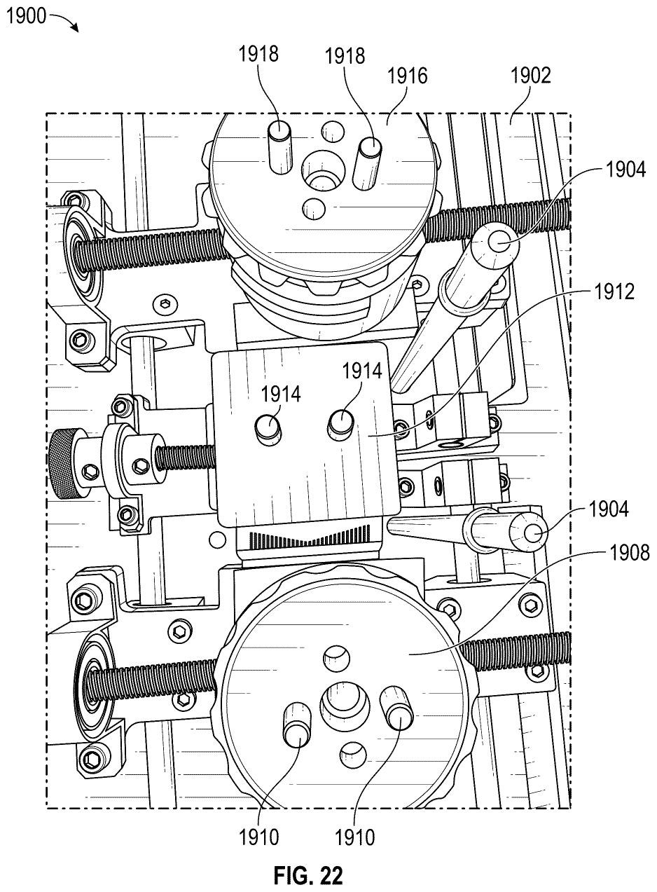

The baseline fixture 1600 may include a base plate 1605 and a sizing abutment 1620. The base plate 1605 may include one or more locating apertures 1610, a sizing aperture 1615, and a plurality of size indicators 1635. The one or more locating apertures 1610 may be configured to receive one or more locating features 1904 (as shown in FIG. 22). The one or more locating apertures 1610 may facilitate alignment and/or positioning of the baseline fixture 1600 relative to the gait modifier outsole 300. The one or more locating apertures 1610 may further facilitate alignment and/or positioning of the baseline fixture 1600 relative to the adjustment platform 1900 (as shown in FIG. 22).

The sizing aperture 1615 may be configured to receive the sizing abutment 1620. The sizing aperture 1615 and the sizing abutment 1620 may be configured to allow the sizing abutment to be positioned relative to the base plate 1605 based on a shoe size and/or foot size of a patient. By adjusting the position of the sizing abutment 1620 based on a patient's foot size and/or a patient's shoe size, a midfoot portion of the patient's foot may be positioned relative to the one or more locating apertures 1610 generally the same as the central portion 315 of the gait modifier outsole 300 is positioned relative to the one or more locating features 1904 (as shown in FIG. 22), with the gait modifier outsole 300 receive on the adjustment platform 1900 (as shown in FIG. 22).

The plurality of size indicators 1635 may correspond to a range of standard shoe sizes. The range of standard shoe sizes may include size 5 through size 15, including half sizes for size 5.5 through size 11.5.

The sizing abutment 1620 may include an abutment aperture 1625 and one or more abutment indicators 1630. The sizing abutment 1620 may be positionable relative to the base plate 1605. The abutment aperture 1625 may be configured to receive a fattener (not shown) to lock the position of the sizing abutment relative to the base plate 1605. The one or more abutment indicators 1630 may be configured to align with one of the plurality of size indicators 1635 based on the position of the sizing abutment 1620 to indicate that the position of the sizing abutment 1620 corresponds to the indicated standard shoe size.

FIG. 18B is a perspective view of a base plate 1640 according to an embodiment of the present disclosure. The baseline fixture 1600 may include the base plate 1640 and the sizing abutment 1620. The base plate 1640 may include similar functions and features previously described for the base plate 1605. The various features of the base plate 1640 with similar reference numbers as those of the base plate 1605 may be similar in function and/or configuration to their counterparts of the base plate 1605. Differences between the base plate 1605 and the base plate 1640 will be set forth below.

The base plate 1640 may include one or more locating apertures 1645, a sizing aperture 1650, a plurality of size indictors 1655, and one or more reference indicators 1660. The one or more locating apertures 1645 may be configured to receive one or more baseline locating features 1704 (as shown in FIG. 20). The one or more locating apertures 1645 may facilitate alignment and/or positioning of the baseline fixture 1600 relative to the gait modifier outsole 300. The one or more locating apertures 1645 may further facilitate alignment and/or positioning of the baseline fixture 1600 relative to the adjustment platform 1700 (as shown in FIG. 20).

The one or more reference indicators 1660 may facilitate proper alignment and/or orientation of the patient's foot relative to the baseline fixture 1600. Proper alignment and/or orientation of the patient's foot may be beneficial by ensure a maximized adjustment range of the gait modifier outsole 300 from the baseline state to the adjusted state.

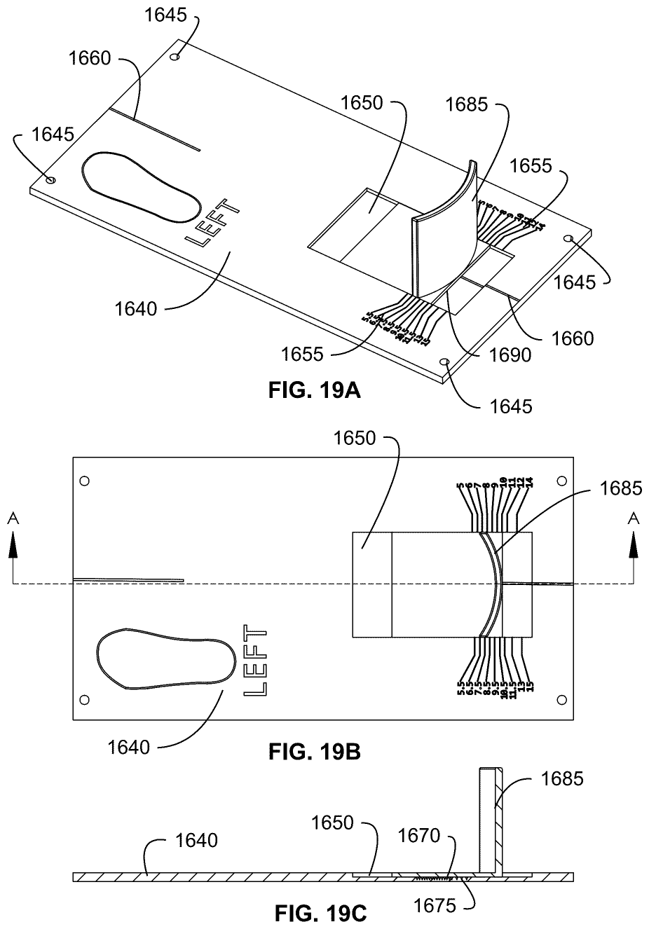

FIG. 19A is a perspective view of a baseline fixture 1600 according to an embodiment of the present disclosure. FIG. 19B is a top view of the baseline fixture 1600 and FIG. 19C is a side section view of the baseline fixture 1600. The baseline fixture 1600 may include a base plate 1640 and a sizing abutment 1685.

The sizing abutment 1685 may include similar functions and features previously described for the sizing abutment 1620. The sizing abutment 1685 may include similar functions and features previously described for the sizing abutment 1620. The sizing abutment 1685 may also include a first locating feature 1670 and an abutment indicator 1690. The abutment indicator 1690 may be configured to align with one of the plurality of size indicators 1655 based on the position of the sizing abutment 1685 to indicate that the position of the sizing abutment 1685 corresponds to the indicated standard shoe size.

The sizing aperture 1650 may include a plurality of second locating features 1675. Each of the plurality of second locating features 1675 may be configured to receive the first locating feature 1670 to secure the position of the sizing abutment 1685 relative to the base plate 1640. Each of the plurality of second locating features 1675 may correspond to one of the plurality of size indicators 1655 so that, with the first locating feature 1670 received in one of the plurality of second locating features 1675, the abutment indicator 1690 aligns with one of the plurality of size indicators 1655 to indicate a standard shoe size.

In an embodiment, the baseline fixture 1600 may be one of a set of differently configured baseline fixtures 1600, one configured for a right foot and one configured for a left foot.

FIG. 20 is a perspective view of an adjustment platform 1700 according to an embodiment of the present disclosure. The adjustment platform 1700 may be configured to receive a gait modifier outsole 300 and facilitate alteration of the gait modifier outsole 300 from a first state to a second state. More specifically, the adjustment platform 1700 may be configured to facilitate alteration of the gait modifier outsole 300 from the neutral state to the baseline state and to the adjusted state. Alternatively, the adjustment platform 1700 may be configured to facilitate alteration of the gait modifier outsole 300 from the neutral state to the adjusted state. Alternatively, the adjustment platform 1700 may be configured to facilitate alteration of the gait modifier outsole 300 from the baseline state to the adjusted state.

The adjustment platform 1700 may be further configured to receive the baseline fixture 1600. The adjustment platform 1700 may be configured to allow adjustment of the gait modifier outsole 300 with the baseline fixture 1600 received on the adjustment platform 1700.

The adjustment platform 1700 and the baseline fixture 1600 may be configured so that, with the gait modifier outsole 300 received on the adjustment platform 1700, and the baseline fixture 1600 received on the adjustment platform 1700, the gait modifier outsole 300 and recorded anatomic features of the baseline fixture 1600 are all visible so that the adjustment platform 1700 may be actuated to adjust the gait modifier outsole 300 to align a portion of the gait modifier outsole 300 with a recorded anatomic feature.

The adjustment platform 1700 may include a plurality of actuation mechanisms, each of which is configured to adjust the relative positions of the hindfoot portion 305 and the forefoot portion 310 of the gait modifier outsole 300. Each of the plurality of actuation mechanisms may function independently of the other actuation mechanisms.

The adjustment platform 1700 may include a first outsole receiving portion 1708, a second outsole receiving portion 1712, and a third outsole receiving portion 1716. The first outsole receiving portion 1708 and the second outsole receiving portion 1712 may each include three degrees of freedom. The third outsole receiving portion 1716 may include one degree of freedom.

The adjustment platform 1700 may further include a platform base 1702, one or more baseline locating features 1704, a central post 1786, and one or more bridge locating features 1706. The platform base 1702 may be sized generally equal to the size of the base plate 1640. All components of the adjustment platform 1700 may be directly and/or indirectly mounted to the platform base 1702, thereby providing a fixed reference for the plurality of actuation mechanisms.

The baseline locating features 1704 may be configured to be received in the locating apertures 1645 of the baseline fixture 1600. The baseline locating features 1704 may facilitate alignment and/or positioning of the baseline fixture 1600 relative to the adjustment platform 1700. Each of the baseline locating features 1704 may include a height adjuster 1788. The height adjuster 1788 may facilitate height adjustment of the baseline fixture 1600 on the adjustment platform 1700 so that the baseline fixture 1600 may be located proximate the gait modifier outsole 300, with the gait modifier outsole 300 received on the adjustment platform 1700.

The central post 1786 may include the bridge locating features 1706. The bridge locating features 1706 may be configured to be received in the platform locating features 1810 of the locating bridge 1800 (as shown in FIG. 21). The bridge locating features 1706 may facilitate alignment and/or positioning of the locating bridge 1800 relative to the adjustment platform 1700.

The first outsole receiving portion 1708 may include one or more first outsole locating features 1710. The first outsole locating features 1710 may be configured to be received in the hindfoot locating apertures 420. The first outsole locating features 1710 and the hindfoot locating apertures 420 may be configured so the rotation of the first outsole receiving portion 1708 results in rotation of the hindfoot portion 405.

The second outsole receiving portion 1712 may include one or more second outsole locating features 1714. The second outsole locating features 1714 may be configured to be received in the forefoot locating apertures 425. The second outsole locating features 1714 and the forefoot locating apertures 425 may be configured so the rotation of the second outsole receiving portion 1712 results in rotation of the forefoot portion 410.

The third outsole receiving portion 1716 may include one or more third outsole locating features 1718. The third outsole locating features 1718 may be configured to be received in the central locating apertures 430. The third outsole locating features 1718 and the central locating apertures 430 may be configured so the linear movement of the third outsole receiving portion 1716 results in linear movement of the central portion 415.

The plurality of actuation mechanisms may include a first actuation mechanism 1720, a second actuation mechanism 1730, a third actuation mechanism 1740, a fourth actuation mechanism 1750, a fifth actuation mechanism 1760, a sixth actuation mechanism 1770, and a seventh actuation mechanism 1780.

The first actuation mechanism 1720, the second actuation mechanism 1730, the fourth actuation mechanism 1750, the fifth actuation mechanism 1760, and the sixth actuation mechanism 1770 may each be configured as a linear actuation mechanism. The third actuation mechanism 1740 and the seventh actuation mechanism 1780 may each be configured a rotational actuation mechanism.

The first actuation mechanism 1720, the second actuation mechanism 1730, and the third actuation mechanism 1740 may be configured to adjust and/or position the hindfoot portion 305. The fourth actuation mechanism 1750 may be configured to adjust and/or position the central portion 315. The fifth actuation mechanism 1760, the sixth actuation mechanism 1770, and the seventh actuation mechanism 1780 may be configured to adjust and/or position the forefoot portion 310.

Alternatively, the first actuation mechanism 1720, the second actuation mechanism 1730, and the third actuation mechanism 1740 may be configured to adjust and/or position the forefoot portion 310. The fifth actuation mechanism 1760, the sixth actuation mechanism 1770, and the seventh actuation mechanism 1780 may be configured to adjust and/or position the hindfoot portion 305.

The first actuation mechanism 1720 may include a first adjustment knob 1722, one or more first guide rails 1724, a first carriage 1726, a first actuation shaft 1728, and a first position indicator 1729. The first actuation mechanism 1720 may be configured to linearly position the first carriage 1726 along an anterior-posterior axis.

The first adjustment knob 1722 may be configured as a user interface to actuate the first actuation mechanism 1720. Rotation of the first adjustment knob 1722 may result in linear motion of the first carriage 1726. The first carriage 1726 may be slidably mounted on the first guide rails 1724. Each of the first guide rails 1724 may be parallel with each other. The first guide rails 1724 may be configured with a circular cross section. Alternatively, the first guide rails 1724 may be configured with a non-circular cross section, such as a square, a rectangle, a dovetail, a trapezoid, or other non-circular geometry.

The first actuation shaft 1728 may be connected to the first adjustment knob 1722 so that rotation of the first adjustment knob 1722 results in rotation of the first actuation shaft 1728. The first actuation shaft 1728 may be configured as a threaded shaft and may be threadably received in the first carriage 1726.

The first position indicator 1729 may indicate a distance and/or a position of the first carriage 1726. The first position indicator 1729 may be configured as a digital readout. The digital readout may have an option to display the distance and/or the position in imperial units and/or metric units. The digital readout may also have an option to reset, or zero, the display to measure a relative distance. Alternatively, the first position indicator 1729 may be configured as a vernier scale, wherein the vernier scale has the option to reset to a zero alignment to measure a relative distance.

The second actuation mechanism 1730 may include a second adjustment knob 1732, a second guide rail 1734, a second carriage 1736, a second actuation shaft 1738, and a second position indicator 1739. The second actuation mechanism 1730 may be configured to linearly position the second carriage 1736 along a medial-lateral axis.

The second adjustment knob 1732 may be configured as a user interface to actuate the second actuation mechanism 1730. Rotation of the second adjustment knob 1732 may result in linear motion of the second carriage 1736. The second carriage 1736 may be slidably mounted on the second guide rail 1734. The second guide rail 1734 may be configured as a single rail with a rectangular cross section.

The second actuation shaft 1738 may be connected to the second adjustment knob 1732 so that rotation of the second adjustment knob 1732 results in rotation of the second actuation shaft 1738. The second actuation shaft 1738 may be configured as a threaded shaft and may be threadably received in the second carriage 1736.

The second position indicator 1739 may indicate a distance and/or a position of the second carriage 1736. The second position indicator 1739 may be configured as a digital readout. The digital readout may have an option to display the distance and/or the position in imperial units and/or metric units. The digital readout may also have an option to reset, or zero, the display to measure a relative distance. Alternatively, the second position indicator 1739 may be configured as a vernier scale, wherein the vernier scale has the option to reset to a zero alignment to measure a relative distance.

The third actuation mechanism may include a third adjustment knob 1742 and a third position indicator 1744. The third adjustment knob 1742 may be configured as a user interface to actuate the third actuation mechanism 1740. Rotation of the third adjustment knob 1742 may result in rotary motion of the first outsole receiving portion 1708. The third position indicator 1744 may indicate a distance and/or a position of the first outsole receiving portion 1708. The third position indicator 1744 may be configured as a vernier scale, wherein the vernier scale has the option to reset to a zero alignment to measure a relative distance. Alternatively, the third position indicator 1744 may be configured as a digital readout. The digital readout may have an option to reset, or zero, the display to measure a relative distance.

The fourth actuation mechanism 1750 may include a fourth adjustment knob 1752, a fourth guide rail 1754, a fourth carriage 1756, a fourth actuation shaft 1758, and a fourth position indicator 1759. The fourth actuation mechanism 1750 may be configured to linearly position the fourth carriage 1756 along a medial-lateral axis.

The fourth adjustment knob 1752 may be configured as a user interface to actuate the fourth actuation mechanism 1750. Rotation of the fourth adjustment knob 1752 may result in linear motion of the fourth carriage 1756. The fourth carriage 1756 may be slidably mounted on the fourth guide rail 1754. The fourth guide rail 1754 may be configured as a single rail with a dovetail cross section.

The fourth actuation shaft 1758 may be connected to the fourth adjustment knob 1752 so that rotation of the fourth adjustment knob 1752 results in rotation of the fourth actuation shaft 1758. The fourth actuation shaft 1758 may be configured as a threaded shaft and may be threadably received in the fourth carriage 1756.

The fourth position indicator 1759 may indicate a distance and/or a position of the fourth carriage 1756. The fourth position indicator 1759 may be configured as a digital readout. The digital readout may have an option to display the distance and/or the position in imperial units and/or metric units. The digital readout may also have an option to reset, or zero, the display to measure a relative distance. Alternatively, the fourth position indicator 1759 may be configured as a vernier scale, wherein the vernier scale has the option to reset to a zero alignment to measure a relative distance.

The fifth actuation mechanism 1760 may include a fifth adjustment knob 1762, one or more fifth guide rails 1764, a fifth carriage 1766, a fifth actuation shaft 1768, and a fifth position indicator 1769. The fifth actuation mechanism 1760 may be configured to linearly position the fifth carriage 1766 along an anterior-posterior axis.

The fifth adjustment knob 1762 may be configured as a user interface to actuate the fifth actuation mechanism 1760. Rotation of the fifth adjustment knob 1762 may result in linear motion of the fifth carriage 1766. The fifth carriage 1766 may be slidably mounted on the fifth guide rails 1764. Each of the fifth guide rails 1764 may be parallel with each other. The fifth guide rails 1764 may be configured with a circular cross section. Alternatively, the fifth guide rails 1764 may be configured with a non-circular cross section, such as a square, a rectangle, a dovetail, a trapezoid, or other non-circular geometry.

The fifth actuation shaft 1768 may be connected to the fifth adjustment knob 1762 so that rotation of the fifth adjustment knob 1762 results in rotation of the fifth actuation shaft 1768. The fifth actuation shaft 1768 may be configured as a threaded shaft and may be threadably received in the fifth carriage 1766.

The fifth position indicator 1769 may indicate a distance and/or a position of the fifth carriage 1766. The fifth position indicator 1769 may be configured as a digital readout. The digital readout may have an option to display the distance and/or the position in imperial units and/or metric units. The digital readout may also have an option to reset, or zero, the display to measure a relative distance. Alternatively, the fifth position indicator 1769 may be configured as a vernier scale, wherein the vernier scale has the option to reset to a zero alignment to measure a relative distance.

The sixth actuation mechanism 1770 may include a sixth adjustment knob 1772, a sixth guide rail 1774, a sixth carriage 1776, a sixth actuation shaft 1778, and a sixth position indicator 1779. The sixth actuation mechanism 1770 may be configured to linearly position the sixth carriage 1776 along a medial-lateral axis.

The sixth adjustment knob 1772 may be configured as a user interface to actuate the sixth actuation mechanism 1770. Rotation of the sixth adjustment knob 1772 may result in linear motion of the sixth carriage 1776. The sixth carriage 1776 may be slidably mounted on the sixth guide rail 1774. The sixth guide rail 1774 may be configured as a single rail with a rectangular cross section.

The sixth actuation shaft 1778 may be connected to the sixth adjustment knob 1772 so that rotation of the sixth adjustment knob 1772 results in rotation of the sixth actuation shaft 1778. The sixth actuation shaft 1778 may be configured as a threaded shaft and may be threadably received in the sixth carriage 1776.

The sixth position indicator 1779 may indicate a distance and/or a position of the sixth carriage 1776. The sixth position indicator 1779 may be configured as a digital readout. The digital readout may have an option to display the distance and/or the position in imperial units and/or metric units. The digital readout may also have an option to reset, or zero, the display to measure a relative distance. Alternatively, the sixth position indicator 1779 may be configured as a vernier scale, wherein the vernier scale has the option to reset to a zero alignment to measure a relative distance.

The seventh actuation mechanism may include a seventh adjustment knob 1782 and a seventh position indicator 1784. The seventh adjustment knob 1782 may be configured as a user interface to actuate the seventh actuation mechanism 1780. Rotation of the seventh adjustment knob 1782 may result in rotary motion of the second outsole receiving portion 1712. The seventh position indicator 1784 may indicate a distance and/or a position of the second outsole receiving portion 1712. The seventh position indicator 1784 may be configured as a vernier scale, wherein the vernier scale has the option to reset to a zero alignment to measure a relative distance. Alternatively, the seventh position indicator 1784 may be configured as a digital readout. The digital readout may have an option to reset, or zero, the display to measure a relative distance.

FIG. 21 is a perspective view of a locating bridge 1800 according to an embodiment of the present disclosure. The locating bridge 1800 may be configured to facilitate alignment and/or positioning of the plate 200 relative to the adjustment platform 1700, so that, with the gait modifier outsole 300 received on the adjustment platform 1700, the plate 200 may be accurately positioned and securably attached to the gait modifier outsole 300.

The various locating features and locating apertures described herein may ensure accurate adjustment of the gait modifier outsole 300 relative to the anatomic features recorded on the baseline fixture 1600, accurate placement of the plate 200 onto the gait modifier outsole 300 thereby creating the gait modifier 395, and accurate placement of the gait modifier 395 onto the gait modifier shoe 100. The end result may be accurate placement of the gait modifier outsole 300 relative to the anatomic features of a patient to provide the most effective treatment.

Additionally, the various locating features and locating apertures described herein also facilitate adjustment of the gait modifier outsole 300 with the gait modifier outsole 300 detached from the gait modifier shoe 100. Further, the gait modifier 395 is detachable and re-attachable from the gait modifier shoe 100 while maintaining the adjusted state of the gait modifier outsole 300.

The locating bridge 1800 may include one or more platform locating features 1810, one or more plate locating features 1820, a base portion, an extension portion 1840, and a plate contacting surface 1850. The base portion 1830 may include the platform locating features 1810 and may have a cross section corresponding to a cross section of the central post 1786. The extension portion 1840 may extend from the base portion 1830. The extension portion 1840 may include the plate locating features 1820 and a plate contacting surface 1850.

The platform locating features 1810 may be configured to receive the bridge locating features 1706. The platform locating features 1810 may facilitate alignment and/or positioning of the locating bridge 1800 relative to the adjustment platform 1700.

The plate locating features 1820 may be configured to receive the locating apertures 210. The plate locating features 1820 may facilitate alignment and/or positioning of the plate 200 relative to the gait modifier outsole 300.

The plate contacting surface 1850 may include a hook-and-loop fastener type surface, (ex: Velcro®). Additionally, or alternatively, the plate contacting surface 1850 may include other means for removably attaching the plate 200 to the locating bridge 1800, such as: magnets, removeable adhesive, and/or another removably and securably attachable fastening mechanism.

FIG. 22 is a perspective view of an adjustment platform 1900 according to an embodiment of the present disclosure. The adjustment platform 1900 may include similar functions and features previously described for the adjustment platform 1700.

The various features of the adjustment platform 1900 with similar reference numbers as those of the adjustment platform 1700 may be similar in function and/or configuration to their counterparts of the adjustment platform 1700. Differences between the adjustment platform 1900 and the adjustment platform 1700 will be set forth below.

The adjustment platform 1900 may include a platform base 1902, one or more locating features 1904, a first outsole receiving portion 1908, one or more first out sole locating features 1910, a second outsole receiving portion 1912, one or more second outsole locating features 1914, a third outsole receiving portion 1916, and one or more third outsole locating features 1918. The adjustment platform 1900 may be configured to receive a gait modifier outsole 300 and facilitate alteration of the gait modifier outsole 300 from a first state to a second state. The adjustment platform 1900 may include a plurality of actuation mechanisms, each of which is configured to adjust the relative positions of the hindfoot portion 305 and the forefoot portion 310 of the gait modifier outsole 300. Each of the plurality of actuation mechanisms may function independently of the other actuation mechanisms.

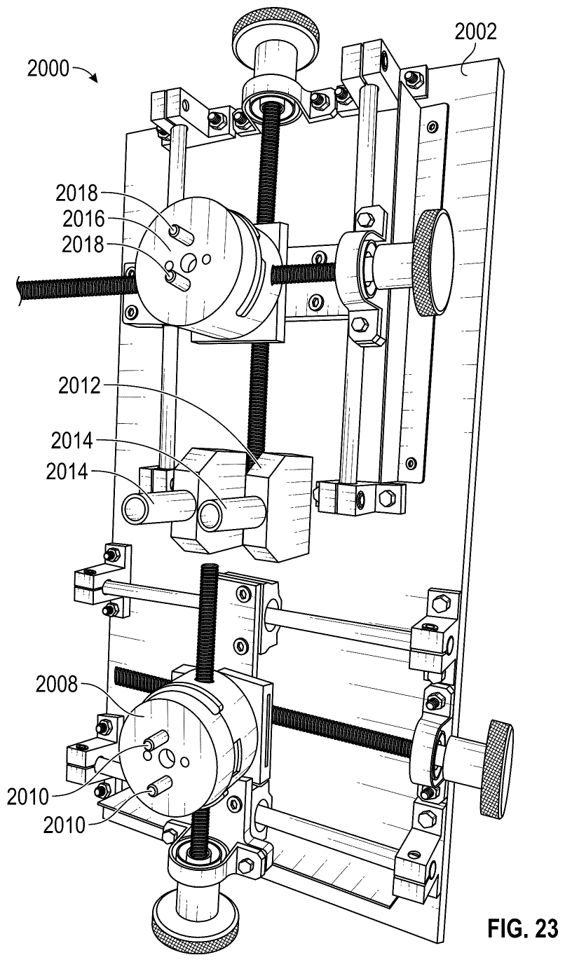

FIG. 23 is a perspective view of an adjustment platform 2000 according to an embodiment of the present disclosure. The adjustment platform 2000 may include similar functions and features previously described for the adjustment platform 1700.

The various features of the adjustment platform 2000 with similar reference numbers as those of the adjustment platform 1700 may be similar in function and/or configuration to their counterparts of the adjustment platform 1700. Differences between the adjustment platform 2000 and the adjustment platform 1700 will be set forth below.

The adjustment platform 2000 may include a platform base 2002, a first outsole receiving portion 2008, one or more first out sole locating features 2010, a second outsole receiving portion 2012, one or more second outsole locating features 2014, a third outsole receiving portion 2016, and one or more third outsole locating features 2018. The adjustment platform 2000 may be configured to receive a gait modifier outsole 300 and facilitate alteration of the gait modifier outsole 300 from a first state to a second state. The adjustment platform 2000 may include a plurality of actuation mechanisms, each of which is configured to adjust the relative positions of the hindfoot portion 305 and the forefoot portion 310 of the gait modifier outsole 300. Each of the plurality of actuation mechanisms may function independently of the other actuation mechanisms.

A system for modifying gait mechanics of a patient may include a gait modifier shoe 100, a plate 200, a gait modifier outsole 300, a baseline fixture 1600, an adjustment platform 1700, and a locating bridge 1800 previously described herein. Alternatively, the system for modifying gait mechanics of a patient may include any alternate embodiments described herein of the above components.

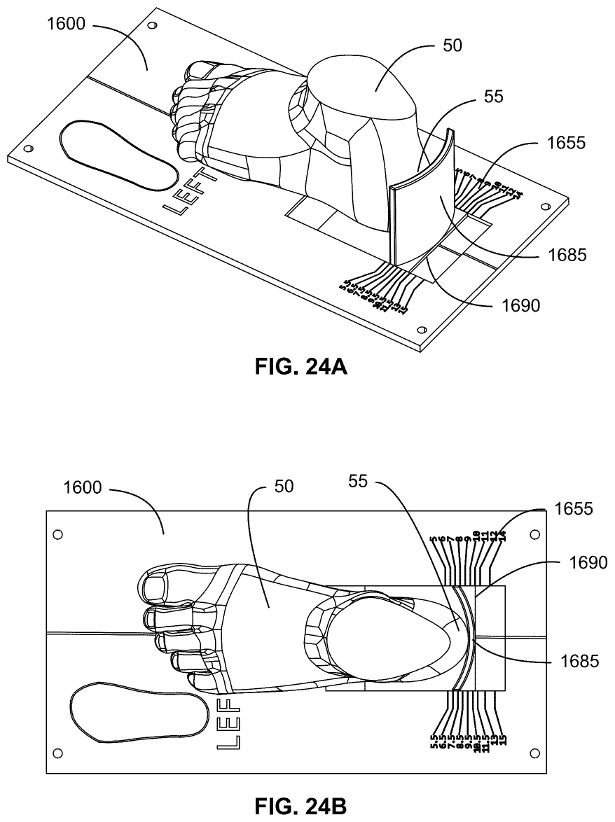

FIG. 24A is a perspective view of an exemplary foot 50 on the baseline fixture 1600 according to an embodiment of the present disclosure. FIG. 24B is a top view of the exemplary foot 50 on the baseline fixture 1600. A method of modifying gait mechanics of a patient may include determining the patient's foot size and/or shoe size. A foot sizer (for example as described in U.S. Pat. No. 1,725,334) may be used to measure a patient's foot size and/or show size. Additionally, or alternatively, a patient may self-report a shoe size.

Starting with either the patient's right foot or the patient's left foot, the appropriate baseline fixture may be selected. The sizing abutment of the baseline fixture may be positioned to align with the patient's shoe size. The patient's foot may be placed on the baseline fixture with the heel 55 abutting the abutment fixture and the foot generally aligned with the reference indicator. A baseline may be measured and marked, using a dry erase marker, or similar marker, the baseline may include, at least, the location of the patient's MTP joint and medial malleolus.

A gait modifier shoe may be selected based on the patient's shoe size. Note: it is recommended that the gait modifier shoe be tested on the patient for fit before therapy to ensure the measure size is accurate and/or appropriate.

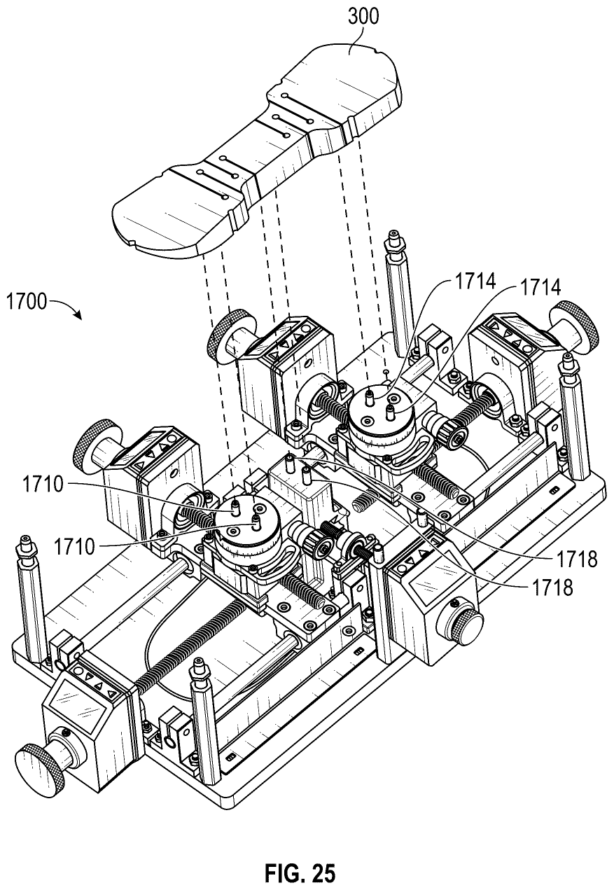

FIG. 25 is a perspective view of a gait modifier outsole 300 and an adjustment platform 1700 according to an embodiment of the present disclosure. A gait modifier outsole may be selected from a set a different gait modifier outsoles based on the patient's shoe size, the baseline measurements, and/or the degree of gait modification desired. The adjustment platform may be adjusted to a neutral state and the gait modifier outsole may be mounted on the adjustment platform.

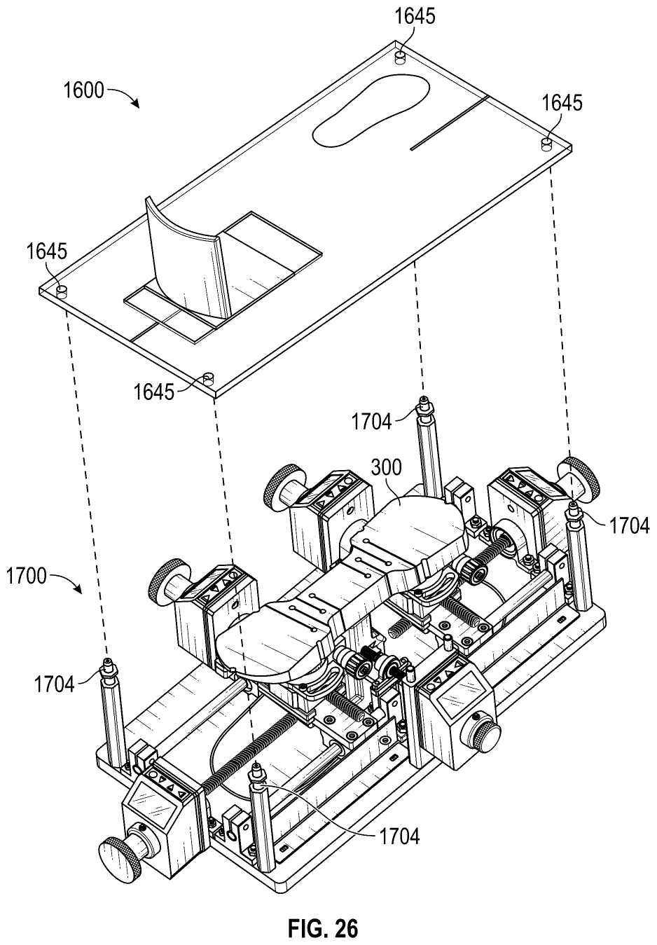

FIG. 26 is a perspective view of the gait modifier outsole 300 and the adjustment platform 1700 and a baseline fixture 1600 according to an embodiment of the present disclosure. The baseline fixture may be mounted on the adjustment platform. Note: the height adjusters of the adjustment platform may be adjusted so that the baseline fixture is proximate the gait modifier outsole. The plurality of actuation mechanisms of the adjustment platform may be actuated to align the hindfoot portion, the forefoot portion, and the central portion of the gist modifier outsole with the markings on the baseline fixture, thereby altering the gait modifier outsole from the neutral state to the baseline state. The positions of each of the plurality of actuation mechanisms may be recorded from the corresponding position indicators and/or digital readouts.

The records of the position indicators and/or digital readouts may allow adjustments and/or positions of the gait modifier outsole 300 to be recreated and/or further adjusted during a future follow-up appointment by the patient. Additionally, or alternatively, the records of the position indicators and/or digital readouts may be used to manufacture a patient specific outsole 301.

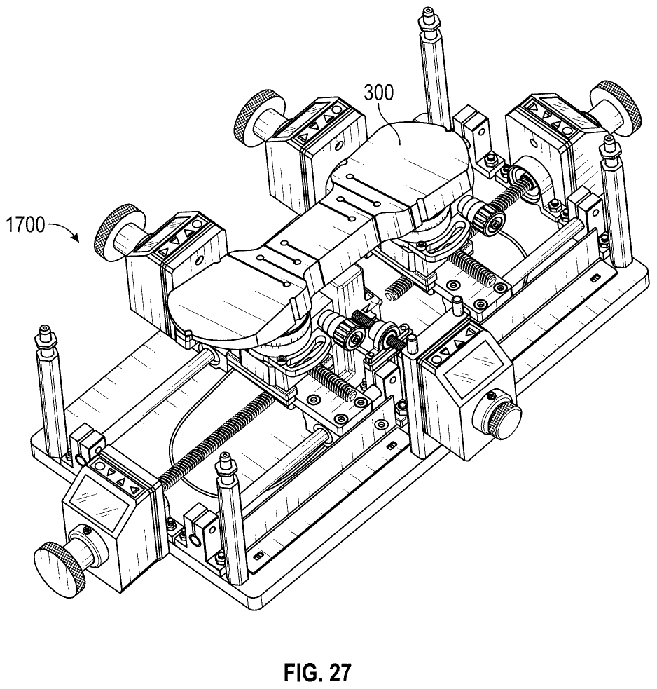

FIG. 27 is a perspective view of the gait modifier outsole 300 and the adjustment platform 1700. The baseline fixture may be removed from the adjustment platform.

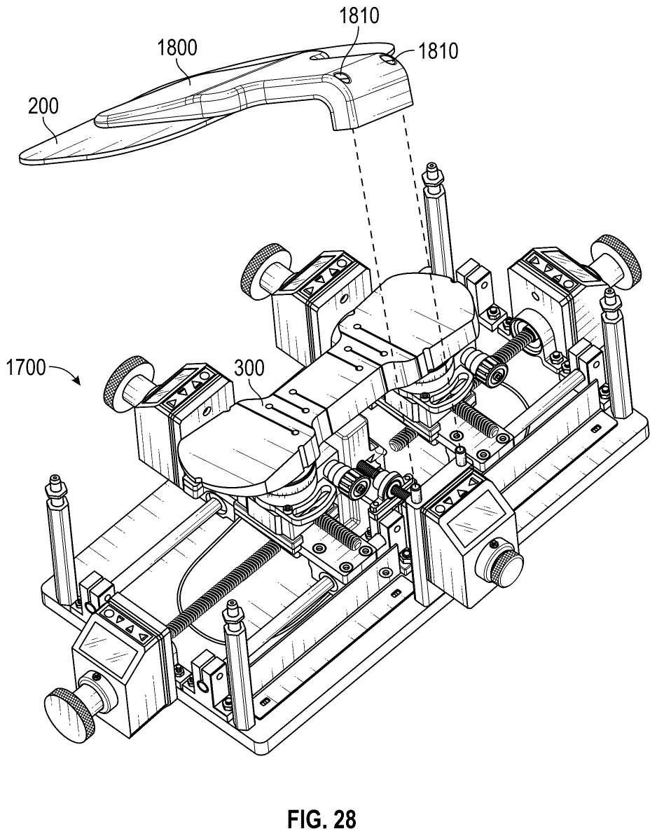

FIG. 28 is a perspective view of the gait modifier outsole 300 and the adjustment platform 1700, a locating bridge 1800, and a plate 200 according to an embodiment of the present disclosure. The plate may be attached to the locating bridge using the appropriate locating features. The locating bridge may be mounted on the adjustment platform using the appropriate locating features.

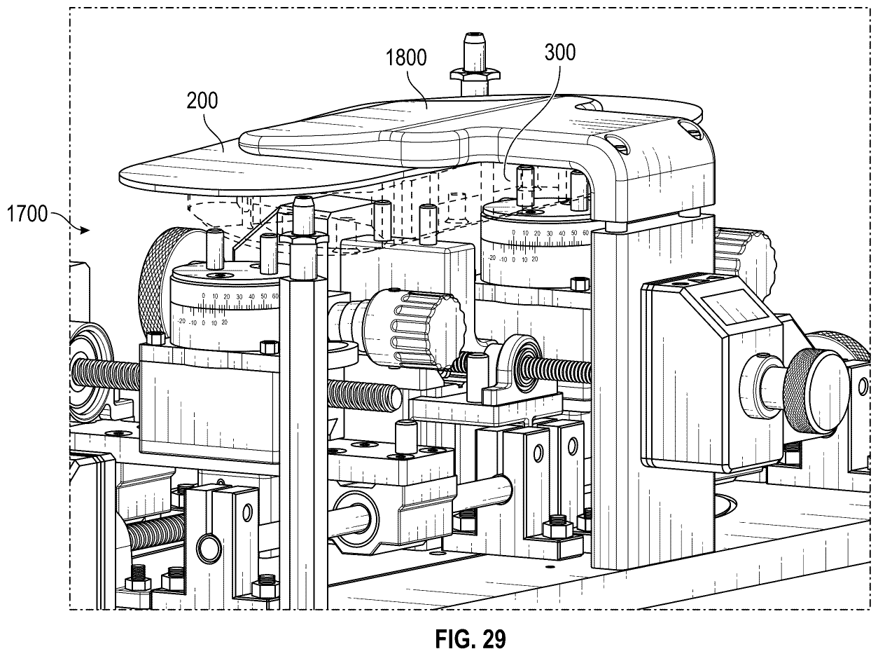

FIG. 29 is a perspective view of the gait modifier outsole 300, the adjustment platform 1700, the locating bridge 1800, and the plate 200. The plate may be securably attached to the gait modifier outsole. The locating bridge may be removed from the adjustment platform, with the gait modifier outsole and plate being removed along with the locating bridge.

The gait modifier outsole and plate, now referred to as the gait modifier, may be removed from the locating bridge. The gait modifier may be placed onto the shoe outsole using the appropriate locating features. All pervious steps may be repeated for the other foot.

With the patient wearing both gait modifier shoes, an initial gait analysis utilizing visual observation and computerized analysis equipment may be performed. Each gait modifier that may need further adjustment may be removed from the gait modifier shoe.

The gait modifier may be re-mounted on the adjustment platform. With the gait modifier outsole properly mounted on the adjustment platform, the plate may be removed from the gait modifier outsole. The gait modifier outsole may be further adjusted using the plurality of actuation mechanisms, thereby altering the gait modifier outsole from the baseline state to the adjusted state based on the results of the gait analysis. The positions of each of the plurality of actuation mechanisms may be recorded from the corresponding position indicators.

The plate may be re-attached to the locating bridge using the appropriate locating features. The locating bridge may be re-mounted on the adjustment platform using the appropriate locating features. The plate may be securably attached to the gait modifier outsole. The locating bridge may be removed from the adjustment platform, with the gait modifier outsole and plate being removed along with the locating bridge. The gait modifier may be removed from the locating bridge. The gait modifier may be placed onto the shoe outsole using the appropriate locating features. A subsequent gait analysis may be performed and the gait modifier adjustment step may be repeated as necessary. The final positions of each of the plurality of actuation mechanisms may be recorded from the corresponding position indicators.

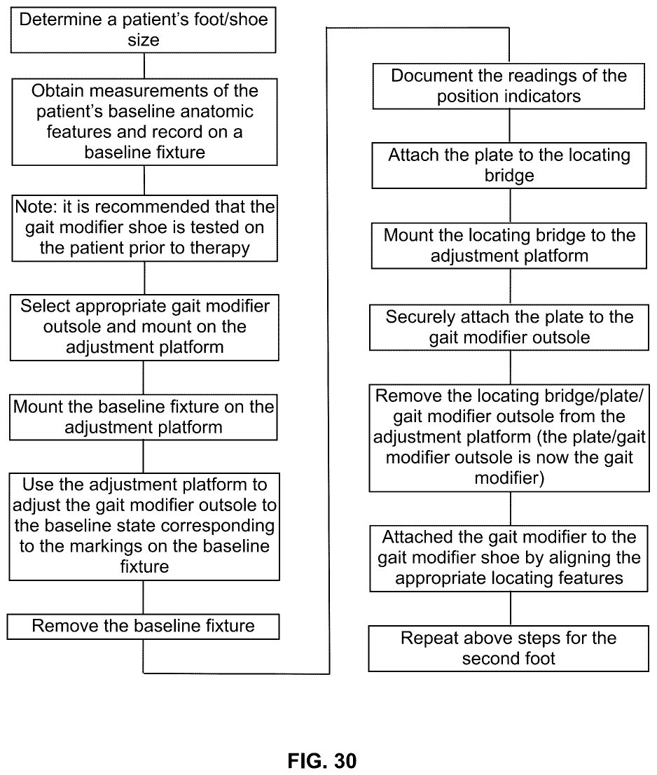

FIG. 30 is an exemplary method for adjusting a gait modifier 395 based on anatomic features according to an embodiment of the present disclosure. An exemplary method for adjusting a gait modifier based on anatomic features may include the following steps:
1. Determine a patient's foot/shoe size.
2. Obtain measurements of the patient's baseline anatomic features and record on a baseline fixture.
   a. Note: It is recommended that the gait modifier shoe is tested on the patient prior to therapy.
3. Select appropriate gait modifier outsole and mount on the adjustment platform.
4. Mount the baseline fixture on the adjustment platform.
5. Use the adjustment platform to adjust the gait modifier outsole to the baseline state corresponding to the markings on the baseline fixture.
6. Remove the baseline fixture.
7. Document the readings of the position indicators and/or digital readouts.
8. Attach the plate to the locating bridge.
9. Mount the locating bridge to the adjustment platform.
10. Securely attach the plate to the gait modifier outsole.
11. Remove the locating bridge/plate/gait modifier outsole from the adjustment platform (the plate/gait modifier outsole is now the gait modifier).
12. Attached the gait modifier to the gait modifier shoe by aligning the appropriate locating features.
13. Repeat above steps for the second foot.

Those of skill in the art will recognize that this is only one of many potential methods that may be used to adjust a gait modifier based on anatomic features. In alternative embodiments, different methods may be used adjust a gait modifier based on anatomic features other than the methods described above. Further, the methods set forth above may be used to adjust a gait modifier based on anatomic features using other devices besides those specifically disclosed herein.

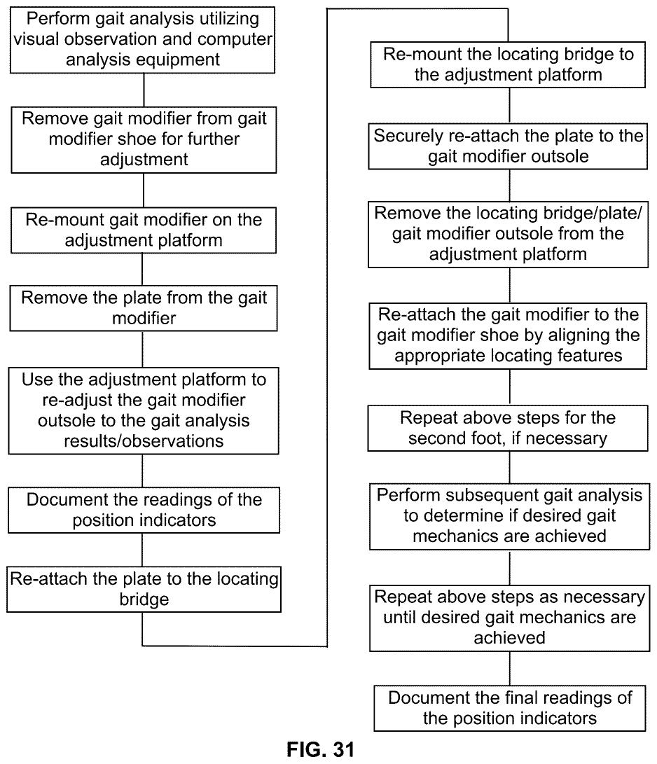

FIG. 31 is an exemplary method for adjusting a gait modifier based on a gait analysis according to an embodiment of the present disclosure. An exemplary method for adjusting a gait modifier based on a gait analysis may include the following steps:
14. Perform gait analysis utilizing visual observation and computer analysis equipment.
15. Remove gait modifier from gait modifier shoe for further adjustment.
16. Re-mount gait modifier on the adjustment platform.
17. Remove the plate from the gait modifier.
18. Use the adjustment platform to re-adjust the gait modifier outsole to the gait analysis results/observations.
19. Document the readings of the position indicators and/or digital readouts.
20. Re-attach the plate to the locating bridge.
21. Re-mount the locating bridge to the adjustment platform.
22. Securely re-attach the plate to the gait modifier outsole.
23. Remove the locating bridge/plate/gait modifier outsole from the adjustment platform.
24. Re-attach the gait modifier to the gait modifier shoe by aligning the appropriate locating features.
25. Repeat above steps for the second foot, if necessary.
26. Perform subsequent gait analysis to determine if desired gait mechanics are achieved.
27. Repeat above steps as necessary until desired gait mechanics are achieved.
28. Document the final readings of the position indicators and/or digital readouts.
29. Manufacturing a patient specific outsole based on the final readings of the position indicators and/or digital readouts and/or a three-dimensional scan of the gait modifier.

Those of skill in the art will recognize that this is only one of many potential methods that may be used to adjust a gait modifier based on a gait analysis. In alternative embodiments, different methods may be used adjust a gait modifier based on a gait analysis other than the methods described above. Further, the methods set forth above may be used to adjust a gait modifier based on a gait analysis using other devices besides those specifically disclosed herein.

An exemplary method for modifying a gait of a patient may include performing step 1 through step 28 as described above. Those of skill in the art will recognize that this is only one of many potential methods that may be used to modify a gait of a patient. In alternative embodiments, different methods may be used to modify the gait of a patient other than the methods described above. Further, the methods set forth above may be used to modify the gait of a patient using other devices besides those specifically disclosed herein.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. A system for modifying gait mechanics of a patient, the system comprising:
a shoe comprising a first outsole; and
a second outsole configured to attach to the first outsole;
wherein:
the second outsole comprises a hindfoot portion, a forefoot portion, and a central portion connecting the hindfoot portion and the forefoot portion;
the hindfoot portion is adjustably positionable relative to the first outsole independently of the forefoot portion; and
the forefoot portion is adjustably positionable relative to the first outsole independently of the hindfoot portion.

2. The system of claim 1, further comprising a plate configured to securely attach to the second outsole and to securely attach to the first outsole.

3. The system of claim 2, wherein, with the plate securely attached to the second outsole, the plate is further configured to prevent adjustment of a first relative position of the hindfoot portion and a second relative position of the forefoot portion.

4. The system of claim 2, wherein the plate and the second outsole are removeable from the first outsole without altering a first position of the hindfoot portion or a second position of the forefoot portion relative to the plate.

5. The system of claim 1, wherein the central portion is adjustably positionable relative to the hindfoot portion and the forefoot portion independently of the hindfoot portion and the forefoot portion.

6. The system of claim 1, wherein the hindfoot portion is linearly adjustably positionable along a first axis and along a second axis, and is rotationally adjustably positionable about a third axis.

7. The system of claim 1, wherein the forefoot portion is linearly adjustably positionable along a first axis and along a second axis, and is rotationally adjustably positionable about a third axis.

8. The system of claim 1, wherein the central portion comprises a generally planar surface between the hindfoot portion and the forefoot portion.

9. The system of claim 8, wherein the generally planar surface contacts ground during ambulation of the patient while wearing the shoe.

10. The system of claim 8, wherein the central portion comprises a plurality of slots configured to facilitate the second outsole to be expanded, contracted, rotated, and/or angled while maintaining a generally planar ground contacting surface.

11. A system for modifying gait mechanics of a patient, the system comprising:
a shoe comprising a first outsole;
a gait modifier comprising a second outsole and a plate; and
wherein:
the second outsole is configured to securably attach to the plate; and
the plate is configured to securably attach to the first outsole so that a position of the second outsole is fixed relative to the first outsole;
wherein the second outsole comprises:
a forefoot portion; and
a hindfoot portion configured to cooperate with the forefoot portion during ambulation of the patient while wearing the shoe to modify the gait mechanics.

12. The system of claim 11, wherein the hindfoot portion comprises three degrees of freedom independent of the forefoot portion, and the forefoot portion comprises three degrees of freedom independent of the hindfoot portion.

13. The system of claim 11, wherein the second outsole further comprises a central portion connecting the hindfoot portion and the forefoot portion.

14. The system of claim 13, wherein the hindfoot portion, the forefoot portion, and the central portion are ground contacting during ambulation.

15. The system of claim 14, wherein at least one of the hindfoot portion, the forefoot portion, and the central portion comprises one or more slots that are not ground contacting.

16. The system of claim 11, wherein the hindfoot portion is adjustably positionable relative to the first outsole independently of the forefoot portion and the forefoot portion is adjustably positionable relative to the first outsole independently of the hindfoot portion.

17. The system of claim 11, wherein, with the plate securely attached to the second outsole, the plate is further configured to prevent adjustment of a first relative position of the hindfoot portion and a second relative position of the forefoot portion.

18. The system of claim 11, wherein the hindfoot portion is linearly adjustably positionable along a first axis and along a second axis, and is rotationally adjustably positionable about a third axis.

19. The system of claim 11, wherein the forefoot portion is linearly adjustably positionable along a first axis and along a second axis, and is rotationally adjustably positionable about a third axis.

20. A system for modifying gait mechanics of a patient, the system comprising:
a shoe comprising a first outsole; and
a gait modifier comprising:
a second outsole; and
a plate;
wherein:
the second outsole is configured to attach to the plate;
the gait modifier is configured to attach to the first outsole;
the second outsole comprises a hindfoot portion positionable at a first position and a first orientation relative to the plate;
the second outsole comprises a forefoot portion comprising a second position and a second orientation relative to the plate;
the first position and the second position are adjustable with the second outsole detached from the plate; and
the first position and the second position are not adjustable with the second outsole attached to the plate.

21. The system of claim 20, wherein the hindfoot portion is adjustably positionable relative to the first outsole independently of the forefoot portion and the forefoot portion is adjustably positionable relative to the first outsole independently of the hindfoot portion.

22. The system of claim 20, wherein the hindfoot portion is linearly adjustably positionable along a first axis and along a second axis, and is rotationally adjustably positionable about a third axis.

23. The system of claim 20, wherein the forefoot portion is linearly adjustably positionable along a first axis and along a second axis, and is rotationally adjustably positionable about a third axis.

24. The system of claim 20, wherein the second outsole further comprises a central portion connecting the hindfoot portion and the forefoot portion.

25. The system of claim 24, wherein the central portion is adjustably positionable relative to the hindfoot portion and the forefoot portion independently of the hindfoot portion and the forefoot portion.

26. The system of claim 24, wherein the central portion comprises a generally planar surface between the hindfoot portion and the forefoot portion.

27. The system of claim 26, wherein the generally planar surface contacts ground during ambulation of the patient while wearing the shoe to modify the gait mechanics.

28. A system for modifying gait mechanics of a patient, the system comprising:
a shoe comprising a first outsole; and
a second outsole configured to attach to the first outsole;
wherein:
the second outsole comprises a hindfoot portion, a forefoot portion, and a central portion connecting the hindfoot portion and the forefoot portion; and
one of the hindfoot portion and the forefoot portion comprises a unidirectional surface configured to inhibit rotation of the second outsole normal to the unidirectional surface during ambulation.

29. The system of claim 28, wherein another of the hindfoot portion and the forefoot portion comprises an omnidirectional surface configured to allow rotation of the second outsole about a longitudinal axis of the second outsole during ambulation.

30. The system of claim 28, wherein both the hindfoot portion and the forefoot portion comprise the unidirectional surface configured to inhibit rotation of the second outsole normal to the unidirectional surface during ambulation.
